(19) United States
(12) Patent Application Publication
(10) Pub. No.: US 2022/0162325 A1
(43) Pub. Date: May 26, 2022

(54) TYPE I INTERFERON-MEDIATED DISORDERS

(71) Applicant: AstraZeneca AB, Sodertalje (SE)

(72) Inventors: Kerry CASEY, Gaithersburg, MD (US); Dominic SINIBALDI, Gaithersburg, MD (US); Michael SMITH, Gaithersburg, MD (US); Miguel SANJUAN, Gaithersburg, MD (US)

(21) Appl. No.: 17/430,801

(22) PCT Filed: Feb. 14, 2020
(86) PCT No.: PCT/EP2020/053962

§ 371 Date: Aug. 13, 2021

Related U.S. Application Data
(60) Provisional application No. 62/806,002, filed on Feb. 15, 2019.

(51) Int. Cl. C07K 16/28; A61P 43/00

(52) U.S. Cl. CPC C07K 16/2866; A61K 2039/505; A61P 43/00

(57) ABSTRACT

The invention provides methods of identifying, diagnosing, treating, and monitoring or prognosing progression of type I IFN-mediated disease or disorder in subjects. The present invention further relates to methods of identifying candidate therapeutic agents for treating a type I interferon-mediated disease or disorder.

The present invention relates to the identification and use of biomarkers for the detection and/or monitoring of a subject suffering from a type I interferon-mediated disease or disorder such as autoimmune diseases (e.g. systemic lupus erythematosus). The invention further relates to corresponding methods of treatment, and to methods for identifying candidate therapeutic agents.

Type I interferon (IFN) signaling drives pathology in a number of autoimmune diseases, in particular in systemic lupus erythematosus (SLE), and can be tracked via type I IFN-inducible transcripts present in whole blood—said transcripts provide a type I IFN gene signature. By way of example, Yao et al. (Hum Genomics Proteomics 2009, pii: 374312) describe the identification of an IFNα/β 21-gene signature and its use as a biomarker of type I IFN-related diseases or disorders.

Said gene signature approach, however, is of limited utility owing to inconsistent correlation between the induced transcript profiles and the corresponding induced protein profiles.

Therefore, there is a need for an alternative, complementary or improved method for detecting and/or monitoring type I IFN activity in a subject.

The present invention solves one or more of the above problems and, for example, provides an accurate and/or robust means for detection of a type 1 IFN-mediated disease or disorder in a subject.

In a first aspect, the invention provides a method of identifying a subject suitable for treatment of a type I interferon-mediated disease or disorder with a therapeutic agent that modulates (e.g. binds) type I interferon activity comprising detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and

wherein the increased level of the first protein and the increased level of the second protein is relative to:
a) the level of the first protein and the level of the second protein, respectively, in a sample from a tissue or person not having the type I interferon-mediated disease or disorder, or
b) the level of one or more control proteins in a sample of the subject.

In a second aspect, the invention provides a method of identifying a subject suitable for treatment of a type I interferon-mediated disease or disorder with a therapeutic agent that modulates (e.g. binds) type I interferon activity comprising:
i) detecting an increased level of a first protein in a sample of the subject and an increased level of a second protein in a sample of the subject,
   wherein the first protein is EPHB2,
   wherein the second protein has a gene expression inducible by type I interferon and displays a Pearson correlation coefficient greater than 0.3 versus a type I interferon gene signature,
   and
   wherein the increased level of the first protein and the increased level of the second protein is relative to:
      a) the level of the first protein and the level of the second protein, respectively, in a sample from a tissue or person not having the type I interferon-mediated disease or disorder,
         or
      b) the level of one or more control proteins in a sample of the subject; and
ii) administering the therapeutic agent.

In a third aspect, the invention provides an anti-type I interferon antibody or an anti-type I interferon receptor antibody that modulates (e.g. binds) type I interferon activity for use in the treatment of a type I interferon-mediated disease or disorder in a subject, wherein the subject has been identified by detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and

wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

In a fourth aspect, the invention provides a method of treating a type I interferon-mediated disease or disorder in a subject, the method comprising administering an anti-type I interferon antibody or an anti-type I interferon receptor antibody that modulates (e.g. binds) type I interferon activity, wherein the subject has been identified by detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and

wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The present invention may further comprise detecting an increased level of at least one other protein in a sample of the subject, wherein the at least one other protein has a gene expression inducible by type I interferon and displays a Pearson correlation coefficient greater than 0.3 versus a type I interferon gene signature; and wherein the increased level of the at least one other protein is relative to:
a) the level of the at least one other protein in a sample from a tissue or person not having the type I interferon-mediated disease or disorder, or
b) the level of one or more control proteins in a sample of the subject.

In a fifth aspect, the invention provides a method of monitoring or prognosing a type I interferon-mediated disease or disorder progression in a subject comprising:
i) identifying a first protein expression level in an initial sample of the subject and at least one other protein expression level in an initial sample of the subject; and
ii) identifying the first protein expression level in a further sample (e.g. taken subsequently in time versus the initial sample) of the subject and the at least one other protein expression level in a further sample of the subject;
   wherein an increase in the expression level of the first protein and an increase in the expression level of the at least one other protein in the further sample relative to the initial sample of the subject prognoses disease progression; or
   wherein a decrease in the expression level of first protein and a decrease in the expression level of the at least one other protein in the further sample relative to the initial sample of the subject prognoses disease regression;
wherein the first protein is EPHB2; and

wherein the at least one other protein has a gene expression inducible by type I interferon and displays a Pearson correlation coefficient greater than 0.3 versus a type I interferon gene signature.

In a sixth aspect, the invention provides a method of monitoring a type I interferon-mediated disease or disorder progression in a subject receiving treatment with a therapeutic agent that modulates (e.g. binds) type I interferon activity comprising:
i) identifying a first protein expression level in an initial sample of the subject and at least one other protein expression level in an initial sample of the subject;
ii) identifying the first protein expression level in a further sample (e.g. taken subsequently in time versus the initial sample) of the subject and the at least one other protein expression level in a further sample of the subject;
iii) administering a therapeutic agent that modulates type I interferon activity to the subject, wherein the therapeutic agent is administered prior to step i) or between steps i) and ii); and
iv) comparing the expression levels of the first protein and the at least one other protein in the initial sample of the subject with the expression levels of the first protein and the at least one other protein, respectively, in the further sample of the subject;
   wherein a variance in the expression levels of the first protein and the at least one other protein indicates a level of efficacy of the therapeutic agent that modulates type I interferon activity;

wherein the first protein is EPHB2; and

wherein the at least one other protein has a gene expression inducible by type I interferon and displays a Pearson correlation coefficient greater than 0.3 versus a type I interferon gene signature.

In a seventh aspect, the invention provides a method of identifying a candidate therapeutic agent for treating a type I interferon-mediated disease or disorder comprising:
i) identifying a first protein expression level in an initial sample of the subject and at least one other protein expression level in an initial sample of the subject;
ii) administering the candidate therapeutic agent to the subject;
iii) identifying the first protein expression level in a further sample (e.g. taken subsequently in time versus the initial sample) of the subject and the at least one other protein expression level in a further sample of the subject; and
iv) comparing the expression levels of the first protein and the at least one other protein in the initial sample of the subject with the expression levels of the first protein and the at least one other protein, respectively, in the further sample of the subject;
   wherein a variance in the expression levels of the first protein and the at least one other protein comprising a reduction in the up-regulation of the first protein and the at least one other protein expression levels, respectively, indicates that the agent is a candidate therapeutic agent;

wherein the first protein is EPHB2; and

wherein the at least one other protein has a gene expression inducible by type I interferon and displays a Pearson correlation coefficient greater than 0.3 versus a type I interferon gene signature.

Throughout the present invention, reference to the second protein and/or the at least one other protein means a protein (each) independently selected from the group consisting of or comprising ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and; SCGF-beta.

In one embodiment, the second protein and/or the at least one other protein forms part of a biological pathway independent from the biological pathway of the first protein.

Preferably, the second protein and/or the at least one other protein are each independently selected from BLC, LAG-3 and IP-10.

When measuring relative expression levels, in one embodiment, the level of at least one of:
the first protein, and
the second protein or the at least one other protein,

is increased by at least 10%.

Alternatively, when measuring relative expression levels, in one embodiment, the average of:
the level of the first protein, and
the level of the second protein and/or the level of the at least one other protein,

is increased by at least 10%.

The therapeutic agent employed may be an anti-type I interferon antibody or an anti-type I interferon receptor antibody. In one embodiment, the therapeutic agent is an anti-type I interferon receptor antibody. Preferably, the anti-type I interferon receptor antibody is anifrolumab. In another embodiment, the therapeutic agent is an anti-type I interferon antibody. Preferably, the anti-type I interferon antibody is sifalumimab.

The subject addressed by the present invention is typically in need of treatment of a type I interferon-mediated disease or disorder selected from systemic lupus erythematosus, discoid lupus, lupus nephritis, dermatomyositis, polymyositis, psoriasis, SSc, vasculitis, sarcoidosis, Sjogren's syndrome, and idiopathic inflammatory myositis. Preferably, the subject is in need of treatment of systemic lupus erythematosus.

Also provided is a method of recording the output (e.g. results) of the methods of the invention on a readable medium.

The following abbreviations are used herein:

DM=dermatomyositis

HD=healthy donor

IBM=inclusion body myositis

IFN=interferon

IFNGS=interferon gene signature

IFNPS=interferon protein signature

LLOQ=lower limit of quantitation

PM=polymyositis

QC=quality control

RBM=rules based medicine

RFU=relative fluorescence units

SLE=systemic lupus erythematosus

SLEDAI=SLE global disease activity index

SOMAmers=slow off-rate modified aptamers

SSc=Systemic Sclerosis

WBC=white blood cell

The present inventors have examined the measurement of circulating proteins, which can infiltrate the bloodstream (e.g. from SLE afflicted tissues) as a tool to detect/monitor global type I IFN activity.

Historically, the presence of anti-DNA autoantibodies in patient serum has prevented effective use of SOMAmers for the evaluation of circulating proteins in SLE. However, the present inventors have adapted protocols to mitigate for those autoantibodies and have reported high reproducibility and accuracy with 100% QC pass rate and have improved correlation with previously validated multi-analyte platform results. Using SOMAmers together with the IFNα/β 21-gene signature (IFNGS) identified by Yao et al. (2009), the present inventors have derived a type I IFN protein signature that can approximate the IFNGS score. This type I IFN protein signature represents a completely new approach for assessing type I IFN activity.

The present invention thus relates to methods of identifying, diagnosing, treating, and monitoring or prognosing disease or disorder progression in subjects. Subjects include any animal having a type I IFN-mediated disease, disorder, or condition. Subjects include any animal having an autoimmune disease or disorder or condition. Subjects include humans, mice, rats, horses, pigs, cats, dogs, and any animal used for research.

The present invention further relates to methods of identifying candidate therapeutic agents.

Identification and Measurement of Proteins Forming the Type I IFNPS

The type I IFN protein signature (IFNPS) of the invention comprises proteins having a gene expression inducible by type I interferon and displaying a Pearson correlation coefficient greater than 0.3 versus a type I interferon gene signature. The type I IFNGS may include the IFNα/β 21-gene signature (IFNGS) identified by Yao et al. (2009). The circulating proteins used in the Pearson correlation may be identified by Somalogic measurement as described in Example 1.

Typically, the Pearson correlation coefficient is greater than 0.1 or greater than 0.15. In one embodiment, the Pearson correlation coefficient is greater than 0.2 or greater than 0.25. In one embodiment, the Pearson correlation coefficient is greater than 0.3 or greater than 0.35. In another embodiment, the Pearson correlation coefficient is greater than 0.4 or greater than 0.45. In another embodiment, the Pearson correlation coefficient is greater than 0.5 or greater than 0.6. In a preferred embodiment, the Pearson correlation coefficient is greater than 0.7.

According to the present invention, the type I IFNPS typically consists of or comprises EPHB2 and one or more of ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; Monocyte Chemotactic Protein 4 (MCP-4); TIMD3; EMR2; Intercellular Adhesion Molecule 1 (ICAM-1); IL-13 Rat Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta.

In one embodiment, the type I IFNPS typically consists of or comprises EPHB2 and one or more of an interferon-inducible chemokine; a dendritic cell/T cell activation marker; or B cell survival/differentiation marker. In an embodiment, the type I IFNPS typically consists of or comprises EPHB2 and a further inflammation/tissue damage & repair marker.

In one embodiment the type I IFNPS typically consists of or comprises EPHB2 and a B cell survival/differentiation marker selected from the group consisting of B cell-activating factor (BAFF), BLC, DLL1, and SLAF7.

In one embodiment the type I IFNPS typically consists of or comprises EPHB2 and BLC.

In one embodiment, the type I IFNPS typically consists of or comprises EPHB2 and a dendritic cell/T cell activation marker selected from the group consisting of AXL Receptor Tyrosine Kinase (AXL), B7-H1, Beta-2-Microglobulin (B2M), SCGF-alpha, TIMD3, Intercellular Adhesion Molecule 1 (ICAM-1), IL-13 Ra1, Interleukin-18 (IL-18), IL-18 BPa, TCCR, IL-3 Ra, LAG-3, LDH-H 1, Glucocorticoid receptor, PARK7, PD-L2, TGF-b R III, TNF-a, CD30 and SCGF-beta.

In one embodiment the type I IFNPS typically consists of or comprises EPHB2 and LAG-3.

In one embodiment, the type I IFNPS typically consists of or comprises EPHB2 and an interferon-inducible chemokine selected from the group consisting of Monocyte Chemotactic Protein 4 (MCP-4), MIP-3b, MCP-1, Macrophage inflammatory protein 3 beta (MIP-3 beta), MCP-3, Fractalkine/CX3CL-1, Interferon gamma Induced Protein 10 (IP-10), Monocyte Chemotactic Protein 2 (MCP-2), Interferon-inducible T-cell alpha chemoattractant (ITAC), and Monokine Induced by Gamma Interferon (MIG).

In one embodiment the type I IFNPS typically consists of or comprises EPHB2 and IP-10.

In one embodiment, the type I IFNPS typically consists of or comprises EPHB2 and a a further inflammation/tissue damage & repair marker selected from the group consisting of ALCAM, Angiopoietin-2 (ANG-2), AREG, C1q, sCD163, CLM6, CD5L, ST4S6, CO8A1, Macrophage Colony-Stimulating Factor 1 (M-CSF), M-CSF R, Cathepsin S, CXCL16, soluble, DERM, EMR2, bFGF, VEGF sR3, PHI, IGFBP-4, Interleukin-1 receptor antagonist (IL-1Ra), JAG1, KYNU, LG3BP, ILT-4, MAPK14, MMP-14, Matrix Metalloproteinase-7 (MMP-7), NAGK, Notch-3, PDGF-CC, PLPP, NADPH-P450 Oxidoreductase, SAA, a1-Antitrypsin, Sialoadhesin, Siglec-7, Osteopontin, BGH3, Tenascin, Tumor necrosis factor receptor 2 (TNFR2), and TS.

In an embodiment, the type I IFNPS consists of or comprises EPHB2, and one or more of BLC, LAG-3 and IP-10.

In an embodiment, the type I IFNPS consists of or comprises EPHB2, BLC, LAG-3 and IP-10.

Reference throughout this specification to a protein (e.g. the first protein, the second protein and the at least one other protein) embraces structurally homologous proteins, such as naturally occurring isoforms or species or allelic variants, and functional equivalents.

Up or Down Regulation of Type I IFN Protein Signature

The present invention provides methods of detecting or identifying type I IFN activity (e.g. level of proteins). These methods of the present invention may employ SOMAmers (slow off-rate modified aptamers) to measure levels of circulating proteins of interest in a sample (i.e. proteins comprised in the type I IFNPS). SOMAmers are short, single-stranded deoxyoligonucleotides selected in vitro from libraries for their ability to bind to discrete molecular targets, modified with functional groups that mimic amino acid side chains. These modifications can interact with more epitopes on a greater range of target molecules, largely as a result of the novel secondary and tertiary structures formed within the SOMAmer reagent itself. In addition, SOMAmers have a low dissociation rate (slow off-rate) with their target. SOMAmers may have higher affinity to and specificity for more diverse proteins, and are less vulnerable to nuclease degradation.

The subject may have a type I IFNPS profile. The type I IFNPS profile may be a strong profile, a moderate profile, or a weak profile. The type I IFNPS profile can readily be designated as strong, moderate, or weak by determining the fold dysregulation of the inducible type I IFNPS profile of the subject (e.g. the fold increase in expression of upregulated type I IFNPS in the subject), relative to a control sample(s) or control subject(s) and comparing the subject's fold dysregulation to that of other subjects having a type I IFNPS profile.

Up or down regulation (e.g. an increased level) of a group of proteins comprised in a type I IFNPS profile can be calculated by well-known methods in the art. The upregulation or downregulation of the type I IFNPS in the subject's signature profile may be by any degree relative to that of a sample from a control (which may be from a sample that is not disease tissue of the subject (e.g. non-lesional skin of a psoriasis subject) or from a person not having the type I interferon-mediated disease or disorder) or may be relative to that of proteins from the subject whose expression is not changed by the disease (control proteins).

The degree of upregulation or downregulation may be at least 10%, at least 15%, at least 20%, at least 25%, at least 30%, at least 40%, at least 50%, at least 60%, at least 70%, at least 75%, at least 80%, at least 85, at least 90%, at least 95%, at least 100%, at least 125%, at least 150%, or at least 200%, or at least 300%, or at least 400%, or at least 500% or more than that of the control or control sample.

A type I IFNPS profile may be calculated as the average fold increase in the expression level of the set of proteins comprised in the protein signature profile. The average fold increase in the expression level of the set of proteins may be between at least about 2 and at least about 15, between at least about 2 and at least about 10, or between at least about 2 and at least about 5. The average fold increase in the expression level of the set of genes may be at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10.

The degree of increased expression level permits the identification of a fold change cutoff for identifying signature positive and signature negative subjects suffering from autoimmune diseases. In one embodiment, the cutoff is at least about 2. In another embodiment, the cutoff is at least about 2.5. In another embodiment, the cutoff is at least about 3. In another embodiment, the cutoff is at least about 3.5. In another embodiment, the cutoff is at least about 4. In another embodiment, the cutoff is at least about 4.5. In another embodiment, the cutoff is chosen from at least 3.5, 3.6, 3.7, 3.8, 3.9, 4.0, 4.1, 4.2, 4.3, 4.4, and 4.5. In another embodiment the cutoff is between about 2 and about 8. In one embodiment, the cutoff is the mean of the increased expression levels of EPHB2 and at least one of BLC, LAG-3 and IP-10. In another embodiment, the cutoff is the median of the increased expression levels of EPHB2 and at least one of BLC, LAG-3 and IP-10.

Furthermore, the subject may overexpress or have a tissue that overexpresses a type I IFN subtype at least 10%, at least 15%, at least 20%, at least 25%, at least 30%, at least 40%, at least 50%, at least 60%, at least 70%, at least 75%, at least 80%, at least 90%, at least 100%, at least 125%, at least 150%, or at least 200%, or at least 300%, or at least 400%, or at least 500% that of the control. The type I IFN subtype may be any one of IFNα1, IFNα2, IFNα4, IFNα5, IFNα6, IFNα7, IFNα8, IFNα10, IFNα14, IFNαI7, IFNα21, IFNβ, or IFNω. The type I IFN subtypes may include all of IFNαI, IFNα2, IFNα8, and IFNαI4.

In one embodiment, the level of at least one of (i) the first protein, and (ii) the second protein or the at least one other protein, has an area under the curve (AUC) in SLE v Healthy Donor (HD) of greater 0.5 relative to:
a) the level of the first protein, or the level of the second protein or the at least one other protein, respectively, in a sample from a tissue or person not having the type I interferon-mediated disease or disorder, or
b) the level of one or more control proteins in a sample of the subject.

In embodiments, the AUC is greater than 0.6. In embodiments, the AUC is greater than 0.7. In embodiments, AUC is greater than 0.8. In embodiments, the AUC is greater than 0.9. In embodiments, the AUC is greater than 0.95. In embodiments, the AUC is greater than 0.975. In embodiments, the AUC is greater than 0.99. In embodiments, the AUC is 1.

In one embodiment, the level of at least one of (i) the first protein, and (ii) the second protein or the at least one other protein is at least one standard deviation from the Healthy Donor Mean relative to:
a) the level of the first protein, or the level of the second protein or the at least one other protein, respectively, in a sample from a tissue or person not having the type I interferon-mediated disease or disorder, or
b) the level of one or more control proteins in a sample of the subject.

In embodiments, the level is at least two standard deviations from the Healthy Donor Mean. In embodiments, the level is at least two standard deviations from the Healthy Donor Mean.

In one embodiment, up or down regulation (e.g. an increased level) is calculated as average fold change in the protein signature expression levels of the group of at least two proteins, wherein the first protein is EPHB2, and wherein the second protein has a gene expression inducible by type I interferon and displays a Pearson correlation coefficient greater than 0.3 versus a type I interferon gene signature (see, e.g. Example 5). Up or down regulation (e.g. an increased level) of a group of proteins is measured relative to:
a) the level of the first protein and the level of the second protein, respectively, in a sample from a tissue or person not having the type I interferon-mediated disease or disorder,
   or
b) the level of one or more control proteins in a sample of the subject.

In embodiments, the average of the level of the first protein, and the level of the second protein and/or the level of the at least one other protein, is increased by at least Y % relative to:
a) the average of the level of the first protein and the level of the second protein, respectively, in a sample from a tissue or person not having the type I interferon-mediated disease or disorder, or
b) the level of one or more control proteins in a sample of the subject.

In embodiments, Y is 10. In embodiments, Y is 15. In embodiments, Y is 20. In embodiments, Y is 25. In embodiments, Y is 30. In embodiments, Y is 40. In embodiments, Y is 50. In embodiments, Y is 60. In embodiments, Y is 70. In embodiments, Y is 80. In embodiments, Y is 90. In embodiments, Y is 100. In embodiments, Y is 125. In embodiments, Y is 150. In embodiments, Y is 200. In embodiments, Y is 300. In embodiments, Y is 400. In embodiments, Y is 500. Preferably, Y is 10. More preferably, Y is 50.

Methods for detecting protein levels include immuno-based assays such as enzyme-linked immunosorbent assays, western blotting, protein arrays, and silver staining.

Up or down regulation of protein levels may be determined by detecting activity of proteins including, but not limited to, detectable phosphorylation activity, de-phosphorylation activity, or cleavage activity.

The sample may be obtained from a subject, from a vendor with patient samples, or a control sample used for calibration or as a control. If the sample is obtained from a subject it may be any biological fluid or tissue, such as whole blood, serum, saliva, urine, synovial fluid, bone marrow, cerebrospinal fluid, nasal secretions, sputum, amniotic fluid, bronchoalveolar lavage fluid, peripheral blood mononuclear cells, total white blood cells, lymph node cells, spleen cells, tonsil cells, or skin. The sample may be obtained by any means known in the art. Preferably, samples are whole blood or serum.

In one embodiment, the level of the first protein and the level of the second protein or the at least one other protein, in a method of the invention, may be detected in the same sample or in different samples. In one embodiment, the level of the first protein and the level of the second protein or the at least one other protein are detected in the same sample. In another embodiment, the level of the first protein and the level of the second protein or the at least one other protein detected are detected in a different samples.

Methods of Identifying, Diagnosing and Treating a Subject

The present invention provides a method of identifying a subject suitable for treatment of a type I interferon-mediated disease or disorder with a therapeutic agent that modulates (e.g. binds) type I interferon activity, said method comprising detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The present invention provides a method of identifying a subject suitable for treatment of a type I interferon-mediated disease or disorder with a therapeutic agent that modulates (e.g. binds) type I interferon activity, said method comprising detecting an increased level of a first protein in a sample of the subject and an increased level of a second protein in a sample of the subject,

wherein the first protein is EPHB2,

wherein the second protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta, and wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The present invention also provides a method of identifying a subject suitable for treatment of a type I interferon-mediated disease or disorder with a therapeutic agent that modulates (e.g. binds) type I interferon activity comprising:
i) detecting an increased level of a first protein in a sample of the subject and an increased level of a second protein in a sample of the subject, wherein the first protein is EPHB2, wherein the second protein has a gene expression inducible by type I interferon and displays a Pearson correlation coefficient greater than 0.3 versus a type I interferon gene signature, and wherein the increased level of the first protein and the increased level of the second protein is relative to:
   a) the level of the first protein and the level of the second protein, respectively, in a sample from a tissue or person not having the type I interferon-mediated disease or disorder,
      or
   b) the level of one or more control proteins in a sample of the subject; and
ii) administering the therapeutic agent.

The present invention provides a method of identifying a subject suitable for treatment of a type I interferon-mediated disease or disorder with a therapeutic agent that modulates (e.g. binds) type I interferon activity comprising:
i) detecting an increased level of a first protein in a sample of the subject and an increased level of a second protein in a sample of the subject, wherein the first protein is EPHB2, wherein the second protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta, and wherein the increased level of the first protein and the increased level of the second protein is relative to:
   a) the level of the first protein and the level of the second protein, respectively, in a sample from a tissue or person not having the type I interferon-mediated disease or disorder,
      or
   b) the level of one or more control proteins in a sample of the subject; and
ii) administering the therapeutic agent.

The present invention further provides an anti-type I interferon antibody or an anti-type I interferon receptor antibody that modulates (e.g. binds) type I interferon activity for use in the treatment of a type I interferon-mediated disease or disorder in a subject, wherein the subject has been identified by detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The present invention provides an anti-type I interferon antibody or an anti-type I interferon receptor antibody that modulates (e.g. binds) type I interferon activity for use in the treatment of a type I interferon-mediated disease or disorder in a subject, wherein the subject has been identified by detecting an increased level of a first protein in a sample of the subject and an increased level of a second protein in a sample of the subject,

wherein the first protein is EPHB2,

wherein the second protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta, and wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The invention also provides a method of treating a type I interferon-mediated disease or disorder in a subject, the method comprising administering an anti-type I interferon antibody or an anti-type I interferon receptor antibody that modulates (e.g. binds) type I interferon activity, wherein the subject has been identified by detecting an increased level of a first protein in a sample of the subject and an increased level of a second protein in a sample of the subject,

wherein the first protein is EPHB2,

wherein the second protein has a gene expression inducible by type I interferon and displays a Pearson correlation coefficient greater than 0.3 versus a type I interferon gene signature, and

wherein the increased level of the first protein and the increased level of the second protein is relative to:
a) the level of the first protein and the level of the second protein, respectively, in a sample from a tissue or person not having the type I interferon-mediated disease or disorder,
   or
b) the level of one or more control proteins in a sample of the subject.

The invention provides a method of treating a type I interferon-mediated disease or disorder in a subject, the method comprising administering an anti-type I interferon antibody or an anti-type I interferon receptor antibody that modulates (e.g. binds) type I interferon activity, wherein the subject has been identified by detecting an increased level of a first protein in a sample of the subject and an increased level of a second protein in a sample of the subject,

wherein the first protein is EPHB2,

wherein the second protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta; and

wherein the increased level of the first protein and the increased level of the second protein is relative to:
a) the level of the first protein and the level of the second protein, respectively, in a sample from a tissue or person not having the type I interferon-mediated disease or disorder, or
b) the level of one or more control proteins in a sample of the subject.

Methods of Monitoring or Prognosing Disease or Disorder Progression

A subject may be monitored for type I IFN-mediated disease or disorder progression by the methods encompassed by the present invention. In particular, the present invention provides a method of monitoring or prognosing a type I interferon-mediated disease or disorder progression in a subject comprising:
i) identifying a first protein expression level in an initial sample of the subject and at least one other protein expression level in an initial sample of the subject; and
ii) identifying the first protein expression level in a further sample (e.g. taken subsequently in time versus the initial sample) of the subject and the at least one other protein expression level in a further sample of the subject;
   wherein an increase in the expression level of the first protein and an increase the expression level of the at least one other protein in the further sample relative to the initial sample of the subject prognoses disease progression; or
   wherein a decrease in the expression level of first protein and a decrease in the expression level of the at least one other protein in the further sample relative to the initial sample of the subject prognoses disease regression;

wherein the first protein is EPHB2; and

wherein the at least one other protein has a gene expression inducible by type I interferon and displays a Pearson correlation coefficient greater than 0.3 versus a type I interferon gene signature.

The present invention provides a method of monitoring or prognosing a type I interferon-mediated disease or disorder progression in a subject comprising:
i) identifying a first protein expression level in an initial sample of the subject and at least one other protein expression level in an initial sample of the subject; and
ii) identifying the first protein expression level in a further sample (e.g. taken subsequently in time versus the initial sample) of the subject and the at least one other protein expression level in a further sample of the subject;
   wherein an increase in the expression level of the first protein and an increase in the expression level of the at least one other protein in the further sample relative to the initial sample of the subject prognoses disease progression; or
   wherein a decrease in the expression level of first protein and a decrease in the expression level of the at least one other protein in the further sample relative to the initial sample of the subject prognoses disease regression;

wherein the first protein is EPHB2; and

wherein the at least one other protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta.

The present invention further provides a method of monitoring a type I interferon-mediated disease or disorder progression in a subject receiving treatment with a therapeutic agent that modulates (e.g. binds) type I interferon activity comprising:
i) identifying a first protein expression level in an initial sample of the subject and at least one other protein expression level in an initial sample of the subject;
ii) identifying the first protein expression level in a further sample (e.g. taken subsequently in time versus the initial sample) of the subject and the at least one other protein expression level in a further sample of the subject;
iii) administering a therapeutic agent that modulates type I interferon activity to the subject, wherein the therapeutic agent is administered prior to step i) or between steps i) and ii); and
iv) comparing the expression levels of the first protein and the at least one other protein in the initial sample of the subject and expression levels of the first protein and the at least one other protein, respectively, in the further sample of the subject;

wherein a variance in the expression levels of the first protein and the at least one other protein indicates a level of efficacy of the therapeutic agent that modulates type I interferon activity;

wherein the first protein is EPHB2; and

wherein the at least one other protein has a gene expression inducible by type I interferon and displays a Pearson correlation coefficient greater than 0.3 versus a type I interferon gene signature.

The present invention provides a method of monitoring a type I interferon-mediated disease or disorder progression in a subject receiving treatment with a therapeutic agent that modulates (e.g. binds) type I interferon activity comprising:
i) identifying a first protein expression level in an initial sample of the subject and at least one other protein expression level in an initial sample of the subject;
ii) identifying the first protein expression level in a further sample (e.g. taken subsequently in time versus the initial sample) of the subject and the at least one other protein expression level in a further sample of the subject;
iii) administering a therapeutic agent that modulates type I interferon activity to the subject, wherein the therapeutic agent is administered prior to step i) or between steps i) and ii); and
iv) comparing the expression levels of the first protein and the at least one other protein in the initial sample of the subject and expression levels of the first protein and the at least one other protein, respectively, in the further sample of the subject;

wherein a variance in the expression levels of the first protein and the at least one other protein indicates a level of efficacy of the therapeutic agent that modulates type I interferon activity;

wherein the first protein is EPHB2; and

wherein the at least one other protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta.

In methods of monitoring or prognosing a type I interferon-mediated disease or disorder progression in a subject, samples from the subject may be obtained at different point in time, e.g. an initial sample and a further sample. Optionally, the samples from the subject may be obtained before and after administration of a therapeutic agent, e.g. an agent that binds to and/or modulates type I IFN activity, or an agent that binds to and does not modulate type I IFN activity, or a combination of agents that may or may not include an agent that binds to and modulates type I IFN activity. Type I IFNPS profiles are obtained in the samples before and after agent administration. The type I IFNPS profiles in the samples are compared.

Comparison may be of the number of proteins of the type I IFNPS present in the samples or may be of the quantity of the proteins of the type I IFNPS present in the samples, or any combination thereof.

Variance prognosing disease progression may be indicated if the number or level (or any combination thereof) of up-regulated proteins of the type I IFNPS increases in the further sample relative to the initial sample.

Variance prognosing disease regression may be indicated if the number or level (or any combination thereof) of up-regulated proteins of the type I IFNPS decreases in the further sample relative to the initial sample.

Variance indicating efficacy of the therapeutic agent may be indicated if the number or level (or any combination thereof) of up-regulated proteins of the type I IFNPS decreases in the sample obtained after administration of the therapeutic agent relative to the sample obtained before administration of the therapeutic agent.

The number of up-regulated proteins of a type I IFNPS may increase or decrease by at least 1, at least 2, at least 3, at least 4, at least 5, at least 6, at least 7, at least 8, at least 9, or at least 10 fold. The level of any given up-regulated protein of a type I IFNPS may decrease by at least 10%, at least 20%, at least 25%, at least 30%, at least 35%, at least 40%, at least 50%, at least 60%, at least 70%, at least 80%, at least 90%, or at least 95%. The number of up-regulated protein of a type I IFNPS with decreased levels may be at least 1, at least 2, at least 3, or at least 4. Any combination of decreased number and decreased level of up-regulated protein of a type I IFNPS may indicate efficacy. Variance indicating efficacy of the therapeutic agent may be indicated if the number or level (or any combination thereof) of down-regulated protein of a type I IFNPS decreases in the sample obtained after administration of the therapeutic agent relative to the sample obtained before administration of the therapeutic agent.

The sample obtained from the subject may be obtained prior to a first administration of the agent, i.e. the subject is naive to the agent. Alternatively, the sample obtained from the subject may occur after administration of the agent in the course of treatment. For example, the agent may have been administered prior to the initiation of the monitoring protocol. Following administration of the agent an additional sample may be obtained from the subject and the type I IFNPS profiles in the samples are compared. The samples may be of the same or different type, e.g. each sample obtained may be a blood sample, or each sample obtained may be a serum sample. The type I IFNPS profiles detected in each sample may be the same, may overlap substantially, or may be similar.

The samples may be obtained at any time before and after the administration of the therapeutic agent.

The sample obtained after administration of the therapeutic agent may be obtained at least 2, at least 3, at least 4, at least 5, at least 6, at least 7, at least 8, at least 9, at least 10, at least 12, or at least 14 days after administration of the therapeutic agent. The sample obtained after administration of the therapeutic agent may be obtained at least 2, at least 3, at least 4, at least 5, at least 6, at least 7, or at least 8 weeks after administration of the therapeutic agent. The sample obtained after administration of the therapeutic agent may be obtained at least 2, at least 3, at least 4, at least 5, or at least 6 months following administration of the therapeutic agent.

Additional samples may be obtained from the subject following administration of the therapeutic agent. At least 2, at least 3, at least 4, at least 5, at least 6, at least 7, at least 8, at least 9, at least 10, at least 12, at least 15, at least 20, at least 25 samples may be obtained from the subject to monitor progression or regression of the disease or disorder over time. Disease or disorder progression may be monitored over a time period of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the subject. Additional samples may be obtained from the subject at regular intervals such as at monthly, bi-monthly, once a quarter year, twice a year, or yearly intervals. The samples may be obtained from the subject following administration of the agent at regular intervals. For instance, the samples may be obtained from the subject at one week following each administration of the agent, or at two weeks following each administration of the agent, or at three weeks following each administration of the agent, or at one month following each administration of the agent, or at two months following each administration of the agent. Alternatively, multiple samples may be obtained from the subject following each administration of the agent.

Disease or disorder progression in a subject may similarly be monitored in the absence of administration of an agent. Samples may periodically be obtained from the subject having the disease or disorder. Disease or disorder progression may be identified if the number up-regulated proteins of a type I IFNPS increases in a later-obtained sample (e.g. further sample) relative to an earlier obtained sample (e.g. initial sample). The number up-regulated proteins of a type I IFNPS may increase by at least 1, at least 2, at least 3, at least 4, at least 5, at least 6, at least 7, at least 8, at least 9, or at least 10. Disease or disorder progression may be identified if level of any given up-regulated proteins of type I IFNPS increases by at least 10%, at least 20%, at least 25%, at least 30%, at least 35%, at least 40%, at least 50%, at least 60%, at least 70%, at least 80%, at least 90%, or at least 95%. Disease or disorder progression may be identified if level of any given down-regulated proteins of a type I IFNPS decreases by at least 10%, at least 20%, at least 25%, at least 30%, at least 35%, at least 40%, at least 50%, at least 60%, at least 70%, at least 80%, at least 90%, or at least 95%. The number of up-regulated proteins of a type I IFNPS with increased levels may be at least 1, at least 2, at least 3, at least 4, at least 5, at least 6, at least 7, at least 8, at least 9, at least 10, at least 15, at least 20, at least 25, at least 30, or at least 35. The number of down-regulated proteins of a type I IFNPS with decreased levels may be at least 1, at least 2, at least 3, at least 4, at least 5, at least 6, at least 7, at least 8, at least 9, at least 10, at least 15, at least 20, at least 25, at least 30, or at least 35. Any combination of increased number and increased level of up-regulated proteins of a type I IFNPS may indicate disease or disorder progression. Alternatively, or in combination, any combination of decreased number and decreased level of down-regulated proteins of a type I IFNPS may indicate disease or disorder progression. Disease or disorder regression may also be identified in a subject having a disease or disorder, not treated by an agent. In this instance, regression may be identified if the number of proteins of a type I IFNPS decreases in a later-obtained sample (e.g. further sample) relative to an earlier obtained sample (e.g. initial sample). The number of proteins of a type I IFNPS may decrease by at least 1, at least 2, at least 3, at least 4, at least 5, at least 6, at least 7, at least 8, at least 9, or at least 10. Disease or disorder regression may be identified if level of any given up-regulated proteins of a type I IFNPS decreases by at least 10%, at least 20%, at least 25%, at least 30%, at least 35%, at least 40%, at least 50%, at least 60%, at least 70%, at least 80%, at least 90%, or at least 95%. Disease or disorder regression may be identified if level of any given down-regulated proteins of a type I IFNPS increases by at least 10%, at least 20%, at least 25%, at least 30%, at least 35%, at least 40%, at least 50%, at least 60%, at least 70%, at least 80%, at least 90%, or at least 95%. The number of up-regulated proteins of a type I IFNPS with decreased levels may be at least 1, at least 2, at least 3, at least 4, at least 5, at least 6, at least 7, at least 8, at least 9, at least 10, at least 15, at least 20, at least 25, at least 30, or at least 35. The number of down-regulated proteins of a type I IFNPS with increased levels may be at least 1, at least 2, at least 3, at least 4, at least 5, at least 6, at least 7, at least 8, at least 9, at least 10, at least 15, at least 20, at least 25, at least 30, or at least 35. Disease or disorder progression, or disease or disorder regression, may be monitored by obtaining samples over any period of time and at any interval. Disease or disorder progression, or disease or disorder regression, may be monitored by obtaining samples over the course of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the subject. Disease or disorder progression, or disease or disorder regression, may be monitored by obtaining samples at least monthly, bi-monthly, once a quarter year, twice a year, or yearly. The samples need not be obtained at strict intervals.

Type I Interferon Mediated Disease, Disorder, or Conditions

A type I IFN mediated disease, disorder, or condition is any that exhibits a type I IFNPS. These diseases, disorders, or conditions include those with an autoimmune component such as systemic lupus erythematosus (SLE), discoid lupus, insulin dependent diabetes mellitus, inflammatory bowel disease (including Crohn's disease, ulcerative colitis, and Celiac's disease), multiple sclerosis, psoriasis, autoimmune thyroiditis, schleroderma, rheumatoid arthritis, glomerulonephritis, idiopathic inflammatory myositis, Sjogren's syndrome, vasculitis, inclusion body myositis (IBM), dermatomyositis (DM), polymyositis (PM), sarcoidosis, scleroderma and lupus nephritis. Other diseases, disorders, or conditions include graft versus host disease and transplant rejection.

In the methods of the present invention, the subject may be in need of treatment of a type I interferon-mediated disease or disorder selected from systemic lupus erythematosus, discoid lupus, lupus nephritis, dermatomyositis, polymyositis, psoriasis, SSc, vasculitis, sarcoidosis, Sjogren's syndrome, and idiopathic inflammatory myositis. Preferably, the subject is need of a treatment of systemic lupus erythematosus.

The subjects may also exhibit any of a number of symptoms as discussed in, e.g. International Publication No. WO 2008/070135, or may have a clinical SLEDAI score or BILAG score as discussed in the same. These symptoms may include fatigue, organ damage, malar rash, and alopecia. The subject may be scored using a known clinical scoring system, e.g. SLEDAI which is an index of SLE disease activity as measured and evaluated within the last 10 days (Bombardier C, Gladman D D, Urowitz M B, Caron D, Chang C H and the Committee on Prognosis Studies in SLE: Derivation of the SLEDAI for Lupus Patients. Arthritis Rheum 35:630-640, 1992.). Disease activity under the SLEDAI scoring system can range from 0 to 105. The following categories of SLEDAI activity have been defined: no activity (SLEDAI=0); mild activity (SLEDAI=1-5); moderate activity (SLEDAI=6-10); high activity (SLEDAI=11-19); very high activity (SLEDAI=20 or higher). (Griffiths et al., Assessment of Patients with Systemic Lupus Erythematosus and the use of Lupus Disease Activity Indices). Another disease scoring index is the BILAG index which is an activity index of SLE that is based on specific clinical manifestations in eight organ systems: general, mucocutaneous, neurological, musculoskeletal, cardiovascular, respiratory, renal, and hematology results. Scoring is based on a letter system, but weighted numerical scores can also be assigned to each letter, making it possible to calculate a BILAG score in the range of 0-72. (Griffiths et al., Assessment of Patients with Systemic Lupus Erythematosus and the use of Lupus Disease Activity Indices). Other scoring indices include the PGA score, the composite responder index (CRI), and the ANAM4™ test.

The methods described herein, e.g. method of identifying a subject suitable for treatment of a type I IFN-mediated disease or disorder, may be used for identifying the subject's disease activity level as measured by any classification methodology known in the art, e.g. mild, moderate, high, or very high.

Therapeutic Agents

A therapeutic agent may be administered to a subject or a subject may be identified as a candidate for administration of an agent or a therapeutic agent. A therapeutic agent may modulate type I interferon activity. Suitable therapeutic agents include molecules that bind to and modulate type I IFN activity. Suitable therapeutic agents include molecules that bind to and modulate activity of receptors of type I interferons. The therapeutic agent may be a small molecule or a biological agent. In embodiments, the therapeutic agent is a small molecule. The small molecule may be synthesised or identified and isolated from a natural source. In other embodiments, the therapeutic agent is a biologic agent. Preferably, the biologic agent is an antibody. The antibody may be an anti-type I interferon antibody or an anti-type I interferon receptor antibody.

The antibody may be an antibody specific for any subtype(s) of type I IFN. For instance, the antibody may be specific for any one of IFNαI, IFNα2, IFNα4, IFNα5, IFNα6, IFNα7, IFNα8, IFNα10, IFNαI4, IFNα17, IFNα21, IFNβ, or IFNω. Alternatively, the antibody may be specific for any two, any three, any four, any five, any six, any seven, any eight, any nine, any ten, any eleven, any twelve type I IFN subtypes. If the antibody is specific for more than one type I IFN subtype, the antibody may be specific for IFNαI, IFNα2, IFNα4, IFNα5, IFNα6, IFNα7, IFNα8, IFNα10, and IFNα21; or it may be specific for IFNαI, IFNα2, IFNα4, IFNα5, IFNα8, and IFNα10; or it may be specific for IFNαI, IFNα2, IFNα4, IFNα5, IFNα8, and IFNα21; or it may be specific for IFNαI, IFNα2, IFNα4, IFNα5, IFNα10, and IFNα21. Antibodies specific for type I IFN include sifalumimab, any biologic or antibody other than sifalumimab, antibodies described in U.S. patent application Ser. Nos. 11/009,410 filed Dec. 10, 2004 and 11/157,494 filed Jun. 20, 2005, 9F3 and other antibodies described in U.S. Pat. No. 7,087,726 (Example 1 and Example 2, those disclosed in Table 3 and Table 4, and/or those disclosed in the table entitled “Deposit of Material” on lines 25-54, column 56), NK-2 and YOK5/19 (WO 84/03105), LO-22 (U.S. Pat. No. 4,902,618), 144 BS (U.S. Pat. No. 4,885,166), and EBI-1, EBI-2, and EBI-3 (EP 119476). A therapeutic agent that modulates IFNa activity may neutralize IFNa activity. One of skill in the art is well aware of preparation and formulation of such biological agents and methods of their administration.

The antibody may be an antibody against a type I interferon receptor, including those disclosed in U.S. Pat. Nos. 7,619,070 and 7,662,381 and International Publication No. WO 2009/100309.

The antibody may be a synthetic antibody, a monoclonal antibody, polyclonal antibodies, a recombinantly produced antibody, an intrabody, a multispecific antibody (including bi-specific antibodies), a human antibody, a humanised antibody, a chimeric antibody, a single-chain Fv (scFv) (including bi-specific scFv), a BiTE molecule, a single chain antibody, a Fab fragments, a F(ab′) fragment, a disulfide-linked Fv (sdFv), or an epitope-binding fragment of any of the above. The antibody may be any of an immunoglobulin molecule or immunologically active portion of an immunoglobulin molecule. Furthermore, the antibody may be of any isotype. For example, it may be any of isotypes IgGI, IgG2, IgG3 or IgG4. The antibody may be a full-length antibody comprising variable and constant regions, or an antigen-binding fragment thereof, such as a single chain antibody, or a Fab or Fab′2 fragment. The antibody may also be conjugated or linked to a therapeutic agent, such as a cytotoxin or a radioactive isotope.

In the methods of treatment with a therapeutic agent (e.g. an anti-type I interferon antibody or an anti-type I interferon receptor antibody that modulates type I interferon activity) or the methods comprising administration of a therapeutic agent, a second agent other than an agent that binds to modulates type I IFN activity, or an agent that binds to and modulates the activity of a receptor of a type I interferon may be administered to the subject. Second agents include, but are not limited to, non-steroidal anti-inflammatory drugs such as ibuprofen, naproxen, sulindac, diclofenac, piroxicam, ketoprofen, diflunisal, nabumetone, etodolac, and oxaprozin, indomethacin; anti-malarial drugs such as hydroxychloroquine; corticosteroid hormones, such as prednisone, hydrocortisone, methylprednisolone, and dexamethasone; methotrexate; immunosuppressive agents, such as azathioprine and cyclophosphamide; and biologic agents that, e.g. target T cells such as Alefacept and Efalizumab, or target TNFa, such as, Enbrel, Remicade, and Humira.

Treatment with the therapeutic agent may result in neutralisation of the type I IFNPS profile. Treatment with the therapeutic agent may result in a decrease in one or more symptoms of the type I IFN-mediated disease or disorder. Treatment with the therapeutic agent may result in fewer flare-ups related to the type I IFN-mediated disease or disorder. Treatment with the agent may result in improved prognosis for the subject having the type I IFN-mediated disease or disorder. Treatment with the agent may result in a higher quality of life for the subject. Treatment with the therapeutic agent may alleviate the need to co-administer second agents or may lessen the dosage of administration of the second agent to the subject. Treatment with the therapeutic agent may reduce the number of hospitalisations of the subject that are related to the type I IFN-mediated disease or disorder.

The therapeutic agent that binds to and modulates type I IFN activity may neutralise a type I IFNPS profile. Neutralisation of the type I IFNPS profile may be a reduction in at least one, at least two, at least three, at least four proteins. Neutralisation of the type I IFNPS profile is a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least two proteins up-regulated in the type I IFNPS profile. Alternatively, neutralisation of the type I IFNPS profile refers to a reduction of expression of up-regulated type I IFNPS proteins that is within at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of those type I IFNPS proteins in a control sample. If the therapeutic agent that binds to and modulates type I IFN activity is a biologic agent, such as an antibody, the agent may neutralise the type I IFN protein signature at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

The therapeutic agent that binds to and modulates type I IFN activity may further or alternatively neutralise expression of one or more type I IFN subtypes. The type I IFN subtypes may include any more than one, more than two, more than three, more than four, more than five, more than six, more than seven, more than eight, more than nine, or more than ten type I IFN subtypes. These subtypes may include IFNαI, IFNα2, IFNα4, IFNα5, IFNα6, IFNα7, IFNα8, IFNα10, IFNαI4, IFNαI7, IFNα21, IFNβ, or IFNω. These subtypes may include all of IFNαI, IFNα2, IFNα8, and IFNαI4. Alternatively, these subtypes may include IFNαI, IFNα2, IFNα4, IFNα5, IFNα8, IFNα10, IFNα21. Neutralisation of the type I IFN subtypes may be a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least five, at least seven, at least eight, or at least ten of the subtypes. Neutralisation of the type I IFN subtypes may be a reduction in expression of type I IFN subtype proteins that is within at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of those type I IFN subtypes in a control sample. If the agent that binds to and modulates type I IFN activity is a biologic agent, such as an antibody, the agent may neutralise the type I IFN subtypes at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

The therapeutic agent that binds to and modulates type I IFN activity may further or alternatively neutralise expression of IFNα receptors, either IFNARI or IFNAR2, or both, or TNFα, or IFNγ, or IFNγ receptors (either IFNGRI, IFNGR2, or both IFNGRI and IFNGR2). Neutralisation of expression of IFNα receptors, either IFNARI or IFNAR2, or both, or TNFα, or IFNγ, or IFNγ receptors (either IFNGRI, IFNGR2, or both IFNGRI and IFNGR2) may be a reduction of at least 2%, at least 3%, at least 4%, at least 5%, at least 7%, at least 8%, at least 10%, at least 15%, at least 25%, at least 30%, at least 35%, at least 40%, at least 45%, at least 50%, at least 60%, at least 70%, at least 75%, at least 80%, or at least 90% of any of the at least one, at least two, at least three, at least five, or at least six of these genes. Neutralisation of expression of IFNα receptors, either IFNARI or IFNAR2, or TNFα, or IFNγ, or IFNγ receptors (either IFNGRI, IFNGR2, or both IFNGRI and IFNGR2) is a reduction of expression of at most 50%, at most 45%, at most 40%, at most 35%, at most 30%, at most 25%, at most 20%, at most 15%, at most 10%, at most 5%, at most 4%, at most 3%, at most 2%, or at most 1% of expression levels of these genes in a control sample. If the therapeutic agent that binds to and modulates type I IFN activity is a biologic agent, such as an antibody, the agent may neutralise expression of IFNα receptors IFNARI or IFNAR2, or TNFα, or IFNγ, or IFNγ receptors IFNGRI or IFNGR2 at doses of 0.3 to 30 mg/kg, 0.3 to 10 mg/kg, 0.3 to 3 mg/kg, 0.3 to 1 mg/kg, 1 to 30 mg/kg, 3 to 30 mg/kg, 5 to 30 mg/kg, 10 to 30 mg/kg, 1 to 10 mg/kg, 3 to 10 mg/kg, or 1 to 5 mg/kg.

Identifying Candidate Therapeutic Agents

A candidate therapeutic for treating a type I IFN-mediated disease or disorder may be identified by the methods encompassed by the present invention. In particular, the present invention provides a method of identifying a candidate therapeutic agent for treating a type I interferon-mediated disease or disorder comprising:
i) identifying a first protein expression level in an initial sample of the subject and at least one other protein expression level in an initial sample of the subject;
ii) administering the candidate therapeutic agent to the subject;
iii) identifying the first protein expression level in a further sample (e.g. taken subsequently in time versus the initial sample) of the subject and the at least one other protein expression level in a further sample of the subject; and
iv) comparing the expression levels of the first protein and the at least one other protein in the initial sample of the subject with the expression levels of the first protein and the at least one other protein, respectively, in the further sample of the subject;

wherein a variance in the expression levels of the first protein and the at least one other protein comprising a reduction in the up-regulation of the first protein and the at least one other protein expression levels, respectively, indicates that the agent is a candidate therapeutic agent;

wherein the first protein is EPHB2; and

wherein the at least one other protein has a gene expression inducible by type I interferon and displays a Pearson correlation coefficient greater than 0.3 versus a type I interferon gene signature.

The present invention also provides a method of identifying a candidate therapeutic agent for treating a type I interferon-mediated disease or disorder comprising:
i) identifying a first protein expression level in an initial sample of the subject and at least one other protein expression level in an initial sample of the subject;
ii) administering the candidate therapeutic agent to the subject;
iii) identifying the first protein expression level in a further sample (e.g. taken subsequently in time versus the initial sample) of the subject and the at least one other protein expression level in a further sample of the subject; and
iv) comparing the expression levels of the first protein and the at least one other protein in the initial sample of the subject with the expression levels of the first protein and the at least one other protein, respectively, in the further sample of the subject;

wherein a variance in the expression levels of the first protein and the at least one other protein comprising a reduction in the up-regulation of the first protein and the at least one other protein expression levels, respectively, indicates that the agent is a candidate therapeutic agent;

wherein the first protein is EPHB2; and

wherein the at least one other protein is selected from ALCAM; Angiopoietin-2 (ANG-2); AREG; C1q; AXL Receptor Tyrosine Kinase (AXL); B cell-activating factor (BAFF); B7-H1; Beta-2-Microglobulin (B2M); sCD163; CLM6; BLC; CD5L; ST4S6; SCGF-alpha; CO8A1; Macrophage Colony-Stimulating Factor 1 (M-CSF); M-CSF R; Cathepsin S; CXCL16, soluble; DLL1; DERM; EPHB2; Monocyte Chemotactic Protein 4 (MCP-4); TIMD3; EMR2; Intercellular Adhesion Molecule 1 (ICAM-1); IL-13 Ra1; Interleukin-18 (IL-18); bFGF; IL-18 BPa; MIP-3b; MCP-1; VEGF sR3; TCCR; PHI; IGFBP-4; IL-3 Ra; LAG-3; Interleukin-1 receptor antagonist (IL-1Ra); JAG1; KYNU; Macrophage inflammatory protein 3 beta (MIP-3 beta); LG3BP; ILT-4; MCP-3; Fractalkine/CX3CL-1; Interferon gamma Induced Protein 10 (IP-10); MAPK14; Monocyte Chemotactic Protein 2 (MCP-2); Interferon-inducible T-cell alpha chemoattractant (ITAC); Monokine Induced by Gamma Interferon (MIG); MMP-14; Matrix Metalloproteinase-7 (MMP-7); NAGK; Notch-3; LDH-H 1; Glucocorticoid receptor; PDGF-CC; PLPP; NADPH-P450 Oxidoreductase; SAA; a1-Antitrypsin; PARK7; Sialoadhesin; Siglec-7; SLAF7; Osteopontin; PD-L2; BGH3; TGF-b R III; TNF-a; CD30; Tenascin; Tumor necrosis factor receptor 2 (TNFR2); TS; and SCGF-beta.

Candidate therapeutics may be any type of molecule including a small molecule or a biological agent. A candidate therapeutic agent identified by the methods encompassed by the present invention may immediately be identified as useful as a therapeutic for a disease, disorder, or condition. Alternatively, a candidate therapeutic agent identified by the methods encompassed by the present invention may need to be further tested and/or modified before selection for treating subjects. Alternatively, a candidate therapeutic agent identified by the methods encompassed by the present invention may, after further testing, be de-selected as a molecule for treating subjects.

In methods that identify candidate therapeutic agents, samples (e.g. cells) comprising a type I IFNPS profile are contacted with an agent. The cells may be any type of cells, such as commercially available immortalised cell lines that comprise a type I IFNPS profile, commercially available immortalised cell lines that have been treated with type I IFN to induce a type I IFNPS profile, cells isolated from a subject having a type I IFNPS profile, or cells isolated from a healthy subject and treated with type I IFN to induce a type I IFNPS profile.

Presence or absence of a change in the type I IFNPS profile of the sample is detected following contacting the sample with the agent. Presence of change may be any change in type I IFNPS profile including at least a 10% decrease in up-regulated expression level of at least 2 proteins of the type I IFNPS profile, at least a 20% decrease of the at least 2 up-regulated proteins, at least a 30% decrease of the at least up-regulated 2 proteins, at least a 40% decrease of the at least 2 up-regulated proteins, at least a 50% decrease of the at least 2 up-regulated proteins, at least a 60% decrease of the at least 2 up-regulated proteins, at least a 70% decrease of the at least 2 up-regulated proteins, at least a 75% decrease of the at least 2 up-regulated proteins, at least an 80% decrease of the at least 2 up-regulated proteins, at least an 85% decrease of the at least 2 up-regulated proteins, at least a 90% decrease of the at least 2 up-regulated proteins, at least a 95% decrease of the at least 2 up-regulated proteins, at least a 96% decrease of the at least 2 up-regulated proteins, at least a 97% decrease of the at least 2 up-regulated proteins, at least a 98% decrease of the at least 2 up-regulated proteins, at least a 99% decrease of the at least 2 up-regulated proteins, or a 100% decrease of the at least 2 up-regulated proteins.

The present invention will now be described in more detail, with reference to the following Figures.

### EXAMPLES

### Example 1

### Somalogic Measurements Using a Mitigated Protocol

The SOMAscan multiplex assay consists of 1.3k individual affinity molecules called SOMAmer® (slow off-rate modified DNA aptamer) reagents, each with very high affinity to their protein targets (Rohloff J C et al., Nucleic Acid Ligands With Protein-like Side Chains: Modified Aptamers and Their Use as Diagnostic and Therapeutic Agents. Mol Ther Nucleic Acids 2014;3:e201; Gold L et al., Aptamer-based multiplexed proteomic technology for biomarker discovery. PLoS One. 2010;5:e15004). Prior to the SOMAscan assay, the biological samples were diluted with a sample diluent containing buffers, salts, detergents and competitors. In the case of SLE samples the competitors were a mixture of SomaLogic Polyanionic Competitor, and single and double stranded herring sperm DNA. The diluted biological samples were incubated for 30 min prior to the addition into each well of a 96 well plate containing a mixture of the 1.3k SOMAmer reagents. After the addition, the sample-SOMAmer reagents mixture was incubated for the formation of affinity complexes.

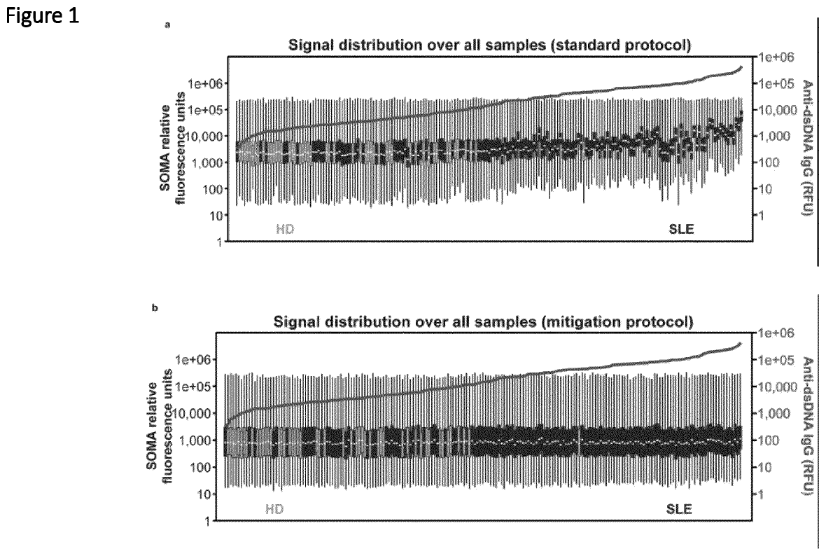

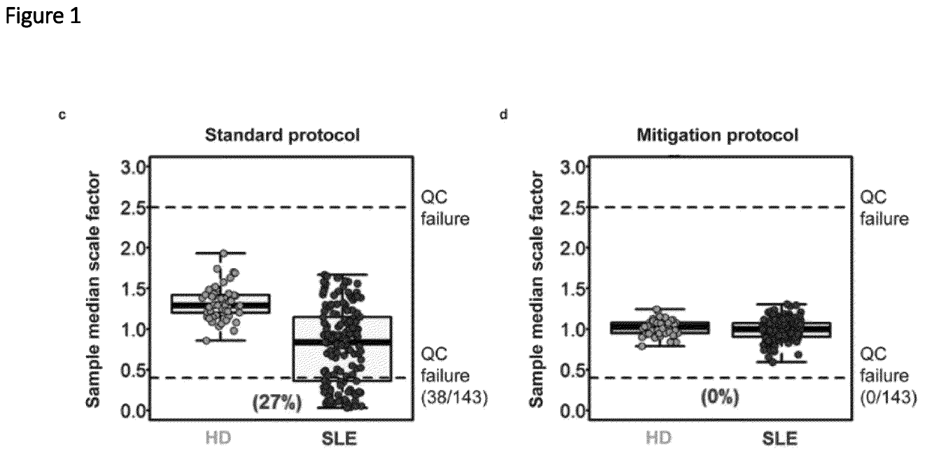

Two sequential bead-based immobilization and washing steps eliminated unbound or non-specifically bound proteins and the unbound SOMAmer reagents, leaving only protein target-bound SOMAmer reagents. These remaining SOMAmer reagents were isolated, and each reagent was quantified simultaneously on a custom Agilent hybridization array. The number of each SOMAmer measured was quantitatively proportional to the protein concentration in the original sample. Somalogic measurements collected with this mitigated protocol passed QC and no longer increased with anti-dsDNA prevalence (FIG. 1).

Data Packaging:

Data standardization procedures were developed to assure data consistency. In the simplest form, normalization procedures controlled for array-to-array variation and were performed in two steps. The first, using a set of hybridization control sequences introduced into the assay eluate prior to hybridization and measured independently for each sample array, which corrected for any systematic effects on the data introduced during the readout phases of the assay. The second normalization scheme used all the SOMAmer signals on a given array to allow for comparison of samples across a plate or within similar groups. It corrected for variation that may be introduced in the course of the SOMAscan assay and natural variation in initial sample concentration that may have occurred. Normalization methods computed scale factors for each sample that was subsequently applied to the signal on the appropriate features within an array. Plate scale and calibration used the endogenous signal from replicate control samples run on each plate and were used to compute scale factors for each plate and for each sequence within a plate to control for plate variation that may have occurred over multiple assay runs.

### Example 2

### Somalogic Measurements Correlated with Rules Based Medicine Measurements

To identify correlates of blood protein Type I IFN activity, a set of protein measurements were identified from Rules Based Medicine (RBM) and SOMAscan platforms that correlated with Type I IFN biology in blood microarray and protein measurements. To identify protein correlates of Type I IFN-dependent gene expression 103 proteins were identified, which correlated with the Type I IFN 21 gene signature with a Spearman Correlation>0.3. To identify proteins associated with IFN-dependent protein prevalence, a blind source separation algorithm was used, Principal Components Analysis followed by a Promax rotation, to identify a score strongly correlated with the Type I IFN 21 gene signature and multiple IFN-inducible chemokine protein measurements. 155 proteins correlated with this score with a Spearman R>0.3. When the union of both lists of proteins was examined, a total of 170 protein measurements were found to have an association with Type I IFN biology.

TABLE 2
Proteins known to have interferon-inducible transcripts as published
within the Interferome database

| Analyte Name | Platform | Gene Symbol | Biological pathway |
|---|---|---|---|
| SOMA-ALCAM | SOMA | ALCAM | inflammation/tissue damage & repair |
| RBM-Angiopoietin-2 | RBM | ANGPT2 | vascular |
| (ANG-2) | | | damage/inflammation/tissue repair |
| SOMA-Angiopoietin-2 | SOMA | ANGPT2 | vascular |
| | | | damage/inflammation/tissue repair |
| SOMA-AREG | SOMA | AREG | vascular |
| | | | damage/inflammation/tissue repair |
| RBM- AXL Receptor | RBM | AXL | dendritic cell/ t cell activation |
| Tyrosine Kinase (AXL) | | | |
| SOMA-b2-Microglobulin | SOMA | B2M | top correlates of IFN21GS |
| RBM-Beta-2-Microglobulin | RBM | B2M | dendritic cell/ t cell activation |
| (B2M) | | | |
| SOMA-C1q | SOMA | C1QA | inflammation/tissue damage & repair |
| RBM-Monocyte | RBM | CCL13 | interferon-inducible chemokine |
| Chemotactic Protein 4 | | | |
| (MCP-4) | | | |
| SOMA-MIP-3b | SOMA | CCL19 | interferon-inducible chemokine |
| SOMA-MCP-1 | SOMA | CCL2 | interferon-inducible chemokine |
| RBM-Monocyte | RBM | CCL2 | interferon-inducible chemokine |
| Chemotactic Protein 1 (MCP-1) | | | |
| RBM-Macrophage | RBM | CCL23 | interferon-inducible chemokine |
| inflammatory protein 3 beta | | | |
| (MIP-3 beta) | | | |
| SOMA-MCP-3 | SOMA | CCL7 | interferon-inducible chemokine |
| RBM-Monocyte | RBM | CCL8 | top correlates of IFN21GS |
| Chemotactic Protein 2 | | | |
| (MCP-2) | | | |
| SOMA-sCD163 | SOMA | CD163 | inflammation/tissue damage & repair |
| SOMA-B7-H1 | SOMA | CD274 | top correlates of IFN21GS |
| SOMA-CLM6 | SOMA | CD300C | inflammation/tissue damage & repair |
| SOMA-CD5L | SOMA | CD5L | inflammation/tissue damage & repair |
| RBM-CD5 Antigen-like | RBM | CD5L | inflammation/tissue damage & repair |
| (CD5L) | | | |
| SOMA-ST4S6 | SOMA | CHST15 | inflammation/tissue damage & repair |
| SOMA-SCGF-alpha | SOMA | CLEC11A | dendritic cell/ t cell activation |
| SOMA-SCGF-beta | SOMA | CLEC11A | dendritic cell/ t cell activation |
| SOMA-CO8A1 | SOMA | COL8A1 | inflammation/tissue damage & repair |
| RBM-Macrophage Colony- | RBM | CSF1 | inflammation/tissue damage & repair |
| Stimulating Factor 1 (M-CSF) | | | |
| SOMA-CSF-1 | SOMA | CSF1 | inflammation/tissue damage & repair |
| SOMA-M-CSF R | SOMA | CSF1R | inflammation/tissue damage & repair |
| SOMA-Cathepsin S | SOMA | CTSS | inflammation/tissue damage & repair |
| SOMA- | SOMA | CX3CL1 | interferon-inducible chemokine |
| Fractalkine/CX3CL-1 | | | |
| RBM-Interferon gamma | RBM | CXCL10 | interferon-inducible chemokine |
| Induced Protein 10 (IP-10) | | | |
| SOMA-IP-10 | SOMA | CXCL10 | interferon-inducible chemokine |
| SOMA-I-TAC | SOMA | CXCL11 | interferon-inducible chemokine |
| RBM-Interferon-inducible | RBM | CXCL11 | interferon-inducible chemokine |
| T-cell alpha | | | |
| chemoattractant (ITAC) | | | |
| SOMA-BLC | SOMA | CXCL13 | B cell survival/differentiation |
| RBM-B Lymphocyte | RBM | CXCL13 | B cell survival/differentiation |
| Chemoattractant (BLC) | | | |
| SOMA-CXCL16, soluble | SOMA | CXCL16 | inflammation/tissue damage & repair |
| RBM-Monokine Induced | RBM | CXCL9 | interferon-inducible chemokine |
| by Gamma Interferon (MIG) | | | |
| SOMA-DLL1 | SOMA | DLL1 | B cell survival/differentiation |
| SOMA-DERM | SOMA | DPT | inflammation/tissue damage & repair |
| SOMA-EMR2 | SOMA | EMR2 | inflammation/tissue damage & repair |
| SOMA-EPHB2 | SOMA | EPHB2 | top correlate of IFN21G5 |
| SOMA-bFGF | SOMA | FGF2 | inflammation/tissue damage & repair |
| SOMA-VEGF sR3 | SOMA | FLT4 | inflammation/tissue damage & repair |
| SOMA-PHI | SOMA | GPI | inflammation/tissue damage & repair |
| SOMA-TIMD3 | SOMA | HAVCR2 | dendritic cell/ t cell activation |
| RBM-Intercellular | RBM | ICAM1 | dendritic cell/ t cell activation |
| Adhesion Molecule 1 | | | |
| (ICAM-1) | | | |
| SOMA-IGFBP-4 | SOMA | IGFBP4 | inflammation/tissue damage & repair |
| SOMA-IL-13 Ra1 | SOMA | IL13RA1 | dendritic cell/ t cell activation |
| RBM-Interleukin-18 (IL-18) | RBM | IL18 | dendritic cell/ t cell activation |
| SOMA-IL-18 BPa | SOMA | IL18BP | dendritic cell/ t cell activation |
| RBM-Interleukin-1 | RBM | IL1RN | inflammation/tissue damage & repair |
| receptor antagonist (IL-1ra) | | | |
| SOMA-TCCR | SOMA | IL27RA | dendritic cell/ t cell activation |
| SOMA-IL-3 Ra | SOMA | IL3RA | dendritic cell/ t cell activation |
| SOMA-JAG1 | SOMA | JAG1 | inflammation/tissue damage & repair |
| SOMA-KYNU | SOMA | KYNU | inflammation/tissue damage & repair |
| SOMA-LAG-3 | SOMA | LAG3 | dendritic cell/ t cell activation |
| SOMA-LDH-H 1 | SOMA | LDHB | dendritic cell/ t cell activation |
| SOMA-LG3BP | SOMA | LGALS3BP | inflammation/tissue damage & repair |
| SOMA-ILT-4 | SOMA | LILRB2 | inflammation/tissue damage & repair |
| SOMA-MAPK14 | SOMA | MAPK14 | inflammation/tissue damage & repair |
| SOMA-MMP-14 | SOMA | MMP14 | inflammation/tissue damage & repair |
| SOMA-MMP-7 | SOMA | MMP7 | inflammation/tissue damage & repair |
| RBM-Matrix | RBM | MMP7 | inflammation/tissue damage & repair |
| Metalloproteinase-7 (MMP-7) | | | |
| SOMA-NAGK | SOMA | NAGK | inflammation/tissue damage & repair |
| SOMA-Notch-3 | SOMA | NOTCH3 | inflammation/tissue damage & repair |
| SOMA-Glucocorticoid | SOMA | NR3C1 | dendritic cell/ t cell activation |
| receptor | | | |
| SOMA-PARK7 | SOMA | PARK7 | dendritic cell/ t cell activation |
| SOMA-PD-L2 | SOMA | PDCD1LG2 | dendritic cell/ t cell activation |
| SOMA-PDGF-CC | SOMA | PDGFC | inflammation/tissue damage & repair |
| SOMA-PLPP | SOMA | PDXP | inflammation/tissue damage & repair |
| SOMA-NADPH-P450 | SOMA | POR | inflammation/tissue damage & repair |
| Oxidoreductase | | | |
| SOMA-SAA | SOMA | SAA1 | inflammation/tissue damage & repair |
| SOMA-a1-Antitrypsin | SOMA | SERPINA1 | inflammation/tissue damage & repair |
| SOMA-Sialoadhesin | SOMA | SIGLEC1 | inflammation/tissue damage & repair |
| SOMA-Siglec-7 | SOMA | SIGLEC7 | inflammation/tissue damage & repair |
| SOMA-SLAF7 | SOMA | SLAMF7 | B cell survival/differentiation |
| RBM-Osteopontin | RBM | SPP1 | inflammation/tissue damage & repair |
| SOMA-BGH3 | SOMA | TGFBI | inflammation/tissue damage & repair |
| SOMA-TGF-b R III | SOMA | TGFBR3 | dendritic cell/ t cell activation |
| SOMA-Tenascin | SOMA | TNC | inflammation/tissue damage & repair |
| SOMA-TNF-a | SOMA | TNF | dendritic cell/ t cell activation |
| RBM-Tumor necrosis | RBM | TNFRSF1B | inflammation/tissue damage & repair |
| factor receptor 2 (TNFR2) | | | |
| SOMA-TNF sR-II | SOMA | TNFRSF1B | inflammation/tissue damage & repair |
| SOMA-CD30 | SOMA | TNFRSF8 | dendritic cell/ t cell activation |
| RBM-B cell-activating | RBM | TNFSF13B | B cell survival/differentiation |
| factor (BAFF) | | | |
| SOMA-BAFF | SOMA | TNFSF13B | B cell survival/differentiation |
| SOMA-TS | SOMA | TYMS | inflammation/tissue damage & repair |

### Example 4

### Independent Confirmation of IFNPS Components by Microarray and Rules Based Medicine (RBM)

As an example of the utility of these 170 proteins, we then set out to identify a small subset that could be used to generate summary score for blood Type I IFN activity. We first filtered the 170 proteins to a list of 87 protein measurements from 75 unique proteins, 20 originating from Rules based Medicine and 67 from SOMAscan platforms, known to have interferon-inducible transcripts as published within the Interferome database. We then used LASSO regression to select a small subset of SOMAscan measurements for use as a summary score. Protein measurements generated in 2013 and 2014 from the NIH-SLE cohort were used as training data to fit the linear model. Protein measurements generated in 2015 were used to validate the model. The shrinkage parameter, λ, and the number of top pearson correlates of the IFN21GS, k, to include in the LASSO model were chosen based on the values that minimized Mean Squared Error (MSE) with the Type I IFN 21 gene signature after 10 iterations of 5-fold cross validation. A linear combination of the top 4 protein correlates of the IFN21GS optimally predicted the IFN21GS in the training set. We refit the model composed of 4 proteins using Ordinary Least Squares (OLS) regression to derive the final coefficient estimates. All protein measurements were log₂ transformed, then scaled to the mean and standard deviation of the respective HD distribution in the training and test sets prior to fitting linear models.

### Example 5

### IFNPS Correlates with SLE Global Disease Activity (SLEDAI)

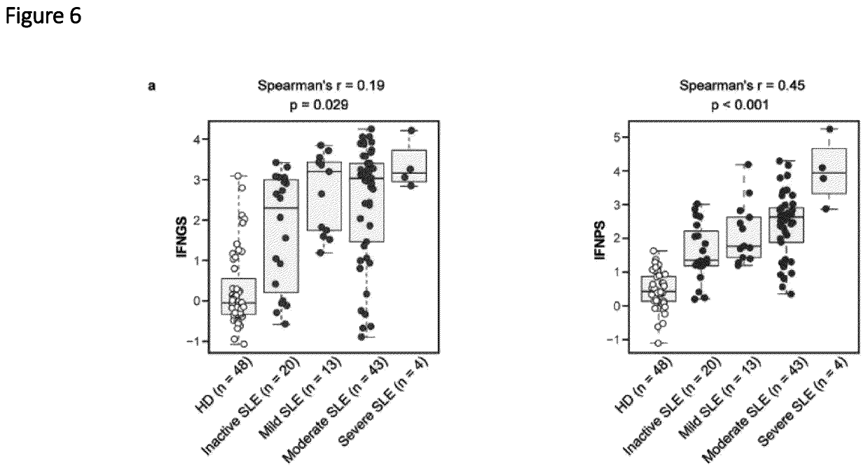

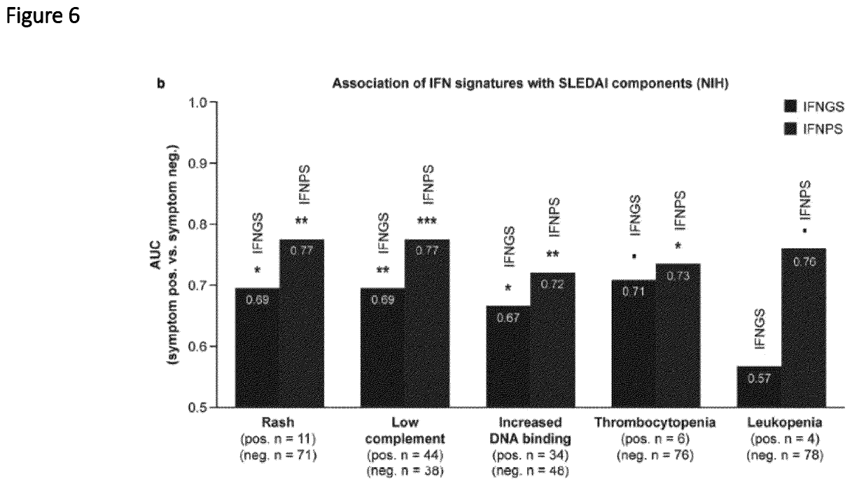

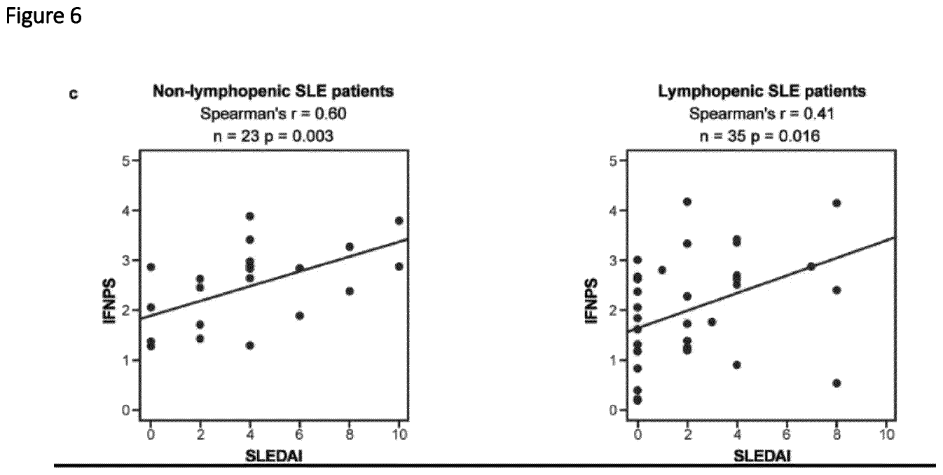

Scatterplots displaying correlation between IFNGS and SLEDAI and 4 protein type I IFN signature and SLEDAI of SLE patients are presented in FIG. 6. AUC of IFNGS and IFNPS discriminated SLE patients with and without specific SLE symptoms (FIG. 6B). Scatterplot displaying relationship between the IFNGS and the 4 protein type I IFN signature in non-lymphopenic and lymphopenic SLE patients are presented in FIG. 6C.

### Example 6

### IFNPS Correlates with SLEDAI in Both Lymphopenic and Non-Lymphopenic SLE Patients

Correlations between IFNGS and SLEDAI and 4 protein type I IFN signature and SLEDAI in non-lymphopenic and lymphopenic SLE patients were obtained, as shown in FIG. 6C.

All statistical analysis was conducted in R 3.1.1. Pairwise correlations were calculated using the non-parametric Spearman's correlation unless otherwise stated. Pairwise comparisons were calculated using the non-parametric Mann-Whitney U Test. The R glmnet package was used to fit the LASSO model.

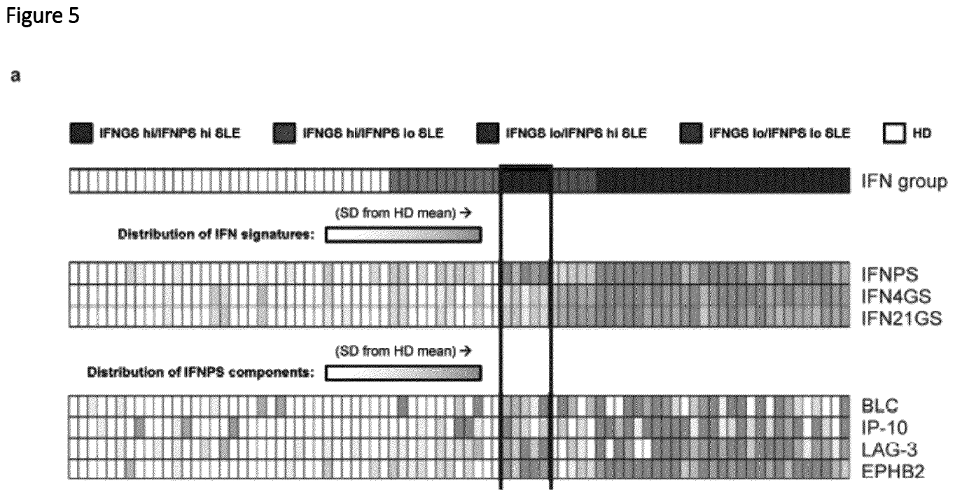

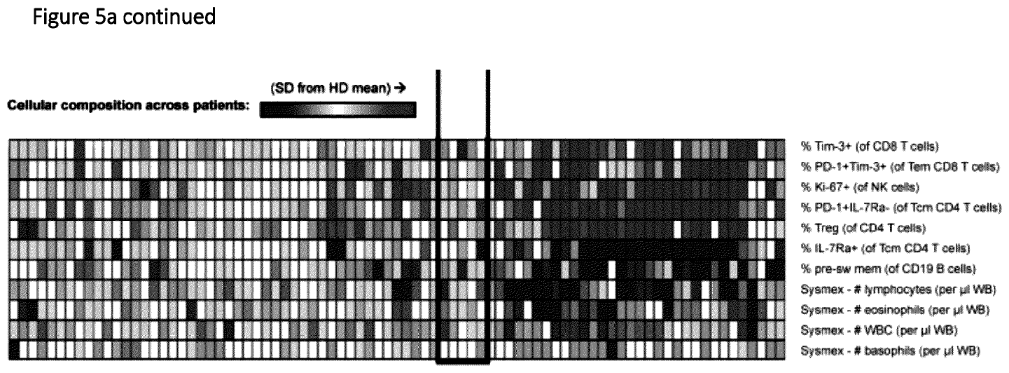

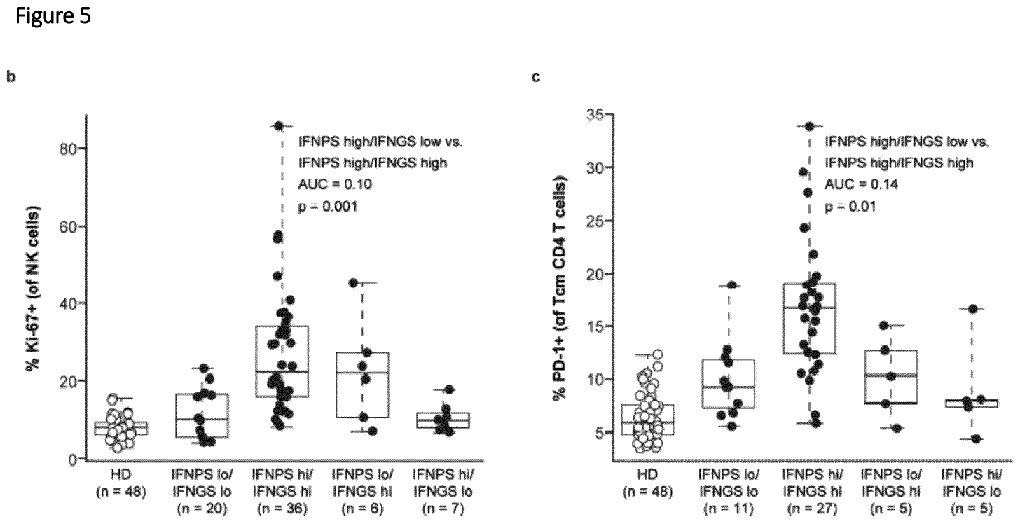

To conclude, in a cohort of 82 SLE patients and 48 healthy donors, the protein signature was found elevated above healthy donors for most of IFNGS test-high patients (49/55, 89%) and also for a subgroup of IFNGS test-low patients (7/27, 26%) (FIG. 5). The protein signature correlated with global disease activity (median SLEDAI score of 4 for the cohort) in both lymphopenic and non-lymphopenic patients (FIG. 6). Significant associations with skin involvement, low complement, anti DNA auto-antibodies, and thrombocytopenia were also observed. In sum, our results suggest blood derived protein measurements can complement validated gene signatures to monitor type I IFN activity.

### Example 7

### IFNPS Identifies a New Subset of Patients with Evidence of Type I IFN Activity

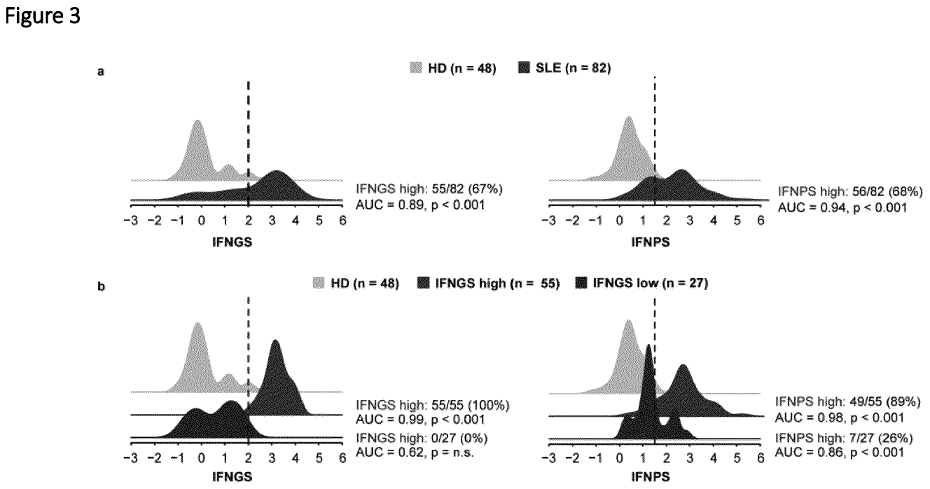

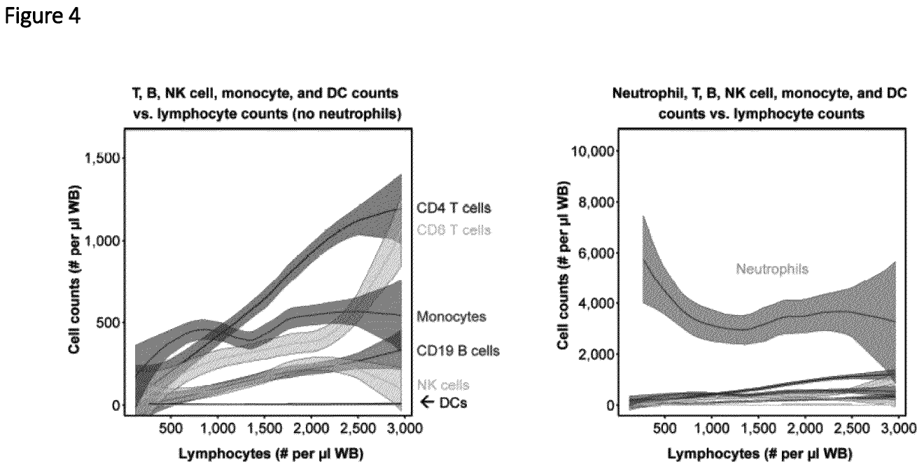

In patients with SLE, the IFNGS displays a bimodal distribution and can be used to separate patients into two subgroups: those with high IFNGS (IFNGS-high) and those with low levels (IFNGS-low). The prevalence of the IFNPS in HD with both IFNGS-high and IFNGS-low patients with SLE was compared. It was found that IFNPS was strongly elevated in all patients with SLE. Of patients with SLE, 68% displayed an IFNPS>2 standard deviations from the HD mean (AUC=0.93, p<0.001). Surprisingly, the IFNPS was also significantly elevated in IFNGS-low patients with SLE (AUC=0.86, p<0.001), and a subset of 26% of IFNGS-low patients with SLE who similarly displayed an IFNPS>2 standard deviations from the HD mean was identified (FIG. 3). IFNPS can therefore be used to identify a unique subset of patients with potentially high type I IFN activity and low levels of IFN-inducible genes in the blood.

### Example 8

### IFNPS and IFNGS Correlate with Global Disease Activity in SLE

The association between the IFNPS and composite disease activity in the training set was characterised to determine if the IFNPS correlates with overall disease activity. The Spearman's correlation of the IFNPS and IFNGS with the SLE Disease Activity Index (SLEDAI) was calculated, an assessment of SLE disease activity across multiple organ systems. It was surprisingly found that IFNPS shared a Spearman's correlation of 0.45 (p<0.001) with SLEDAI, while the IFNGS displayed a Spearman's correlation of 0.19 (p=0.029) with SLEDAI, suggesting the IFNPS could serve as a useful biomarker of composite disease in SLE (FIG. 6A).

The prevalence of the IFNPS and IFNGS in patients positive and negative for each SLEDAI component was examined. Both the IFNGS and IFNPS were significantly elevated in patients who presented with rash, low complement and anti-dsDNA autoantibodies. The IFNPS was also significantly elevated in thrombocytopenic patients with SLE, and the IFNGS displayed a similar trend. The IFNPS also displayed numerical elevation in leukopenic patients with SLE (FIG. 6B). The IFNPS significantly correlates with SLEDAI in both lymphopenic and non-lymphopenic patients with SLE (p<0.05), providing further evidence that the signature reflects tissue biology that is insensitive to blood compositional changes (FIG. 6c).

To determine whether these findings could be reproduced in an independent cohort of patients with moderate to severe disease, the association between the IFNPS and SLEDAI composite score in both lymphopenic and non-lymphopenic patients in the MUSE cohort was assessed. There was significant association between IFNPS and hypocomplementemia, increased anti-dsDNA, and leukopenia in this cohort ( ). In two thrombocytopenic patients The IFNPS displayed a positive correlation with Cutaneous Lupus Erythematosus Disease Area and Severity Index (CLASI) activity score (Spearman's r=0.21, p<0.001), an alternative measure of cutaneous disease activity, confirming the association between IFNPS and SLE cutaneous involvement.

In summary, the IFNPS reflects inflammation across multiple organ systems in patients with SLE, making the IFNPS a surprisingly useful biomarker of composite disease activity.

### Example 9

### IFNPS is Associated with the Type I IFN Pathway

Type I and type II IFNs have distinct roles in amplifying immune response but induce largely overlapping transcriptional changes in cells. Moreover, type II IFNs are directly inducible by type I IFNs. For these reasons, distinguishing between both types of responses while monitoring human disease is challenging. To verify that the IFNPS we identified is reflecting type I IFN—and not type II IFN-associated biology, the correlation between IFNPS and transcription of several components of IFN-γ-inducible gene signatures, IRF1, CXCL9, and SLAMF8,39,41 was measured, and therefore was found to be no correlation between the IFNPS and these genes in samples from patients with either SLE or myositis. In contrast, the IFNPS correlated with all four components of a type I IFN-inducible gene signature, IFI44L, IFI27, RSAD2, and IFI44, demonstrating that the IFNPS is directly induced by type I IFNs and not type II IFNs.

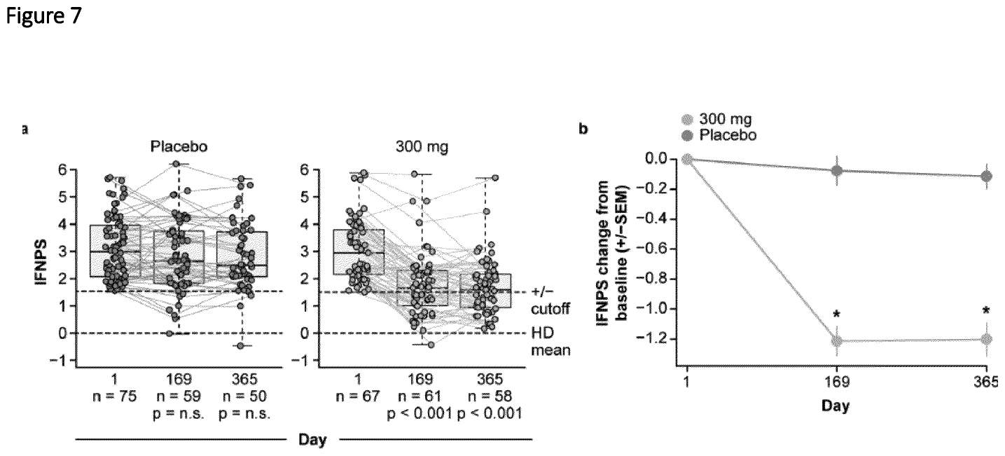

To further evaluate whether the IFNPS is specifically induced by type I IFNs, we investigated whether the IFNPS is suppressed by neutralisation of IFNAR, the receptor necessary for type I IFN signalling. The IFNPS of patients with SLE before and after treatment with anifrolumab, a monoclonal antibody that neutralises IFNAR, was monitored. We found that the IFNPS was significantly decreased at Days 169 and 365 compared with Day 1 (p<0.001) in the anifrolumab 300-mg treatment group. In contrast, the IFNPS displayed no significant changes from baseline in the placebo group (p>0.05; FIG. 7a). Changes in the IFNPS from Day 1 to Days 169 and 365 were also significant when compared between the anifrolumab 300-mg group and placebo group (FIG. 7b). Therefore, the IFNPS was specifically suppressed following IFNAR neutralisation with anifrolumab, demonstrating that the IFNPS reflects biology induced by type I IFN.

FIG. 1: Circulating proteins provide largely distinct data from that found in whole blood gene expression. Density plots displaying spearman correlation of paired RBM and HGU133 Plus 2.0 measurements in 50 HD and 143 SLE samples. Analysis limited to RBM analytes where 75% of measurements were above LLOQ in the specific sample group. Somalogic measurements collected with new mitigated protocol pass QC and no longer increase with anti-dsDNA prevalence. Boxplots displaying global signal distribution of 1129 protein measurements generated using the standard Somalogic protocol (A) and mitigation protocol (B) from serum samples isolated from 143 SLE samples and 50 HD. The min, first quartile, median, third quartile, and max RFU per sample are indicated on each boxplot. Anti-dsDNA prevalence for each sample is plotted in green C. Sample specific median scaling factors used for plate median normalisation in SLE and HD. Samples with a sample scaling factor less than 0.4 signifying a median RFU 2.5 times greater than plate median RFU failed quality control.

Mitigated protocol improves correlation with Rules Based Medicine measurements. Density plots displaying spearman correlation of paired RBM and Somalogic measurements in the 50 HD, anti-dsDNA− SLE samples, and anti-dsDNA+ SLE samples generated from both the standard and mitigated protocol. Only RBM analytes where 75% of measurements were above LLOQ in the specific sample group were used in correlation analysis.

Majority of analytes display high reproducibility. A. Reproducibility of RFU of samples run within same plate on same day (A) and on different plates on different days (B) under mitigation protocol. Boxplots represent the 10^{th} and 90^{th} percentile, interquartile range, and median distribution of CVs among the three replicate experiments of the HD, anti-dsDNA− SLE, and anti-dsDNA+ SLE sample.

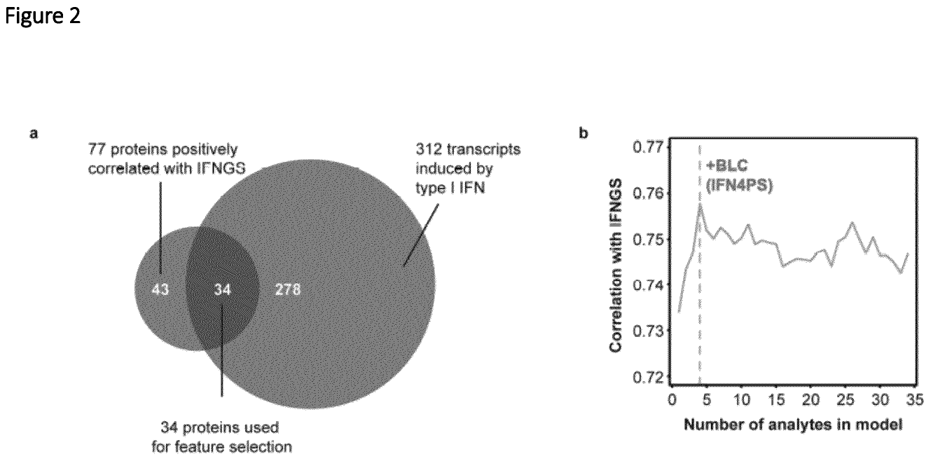

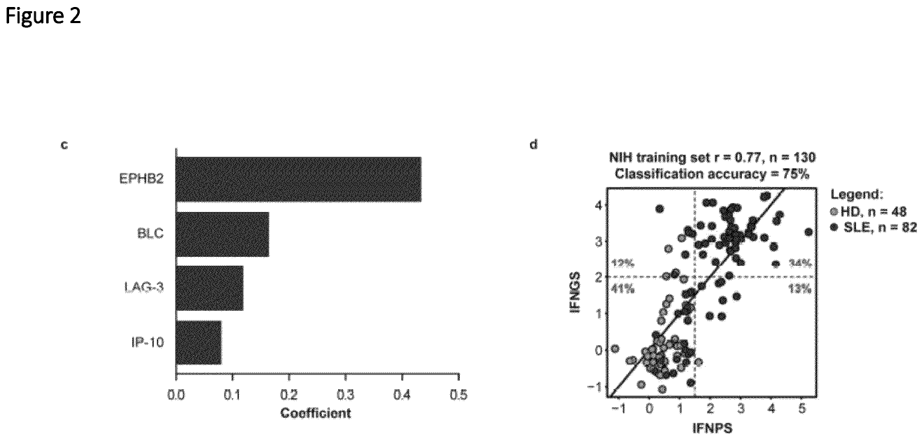

FIG. 2: Identification of an Interferon Protein Signature (IFNPS). A. Venn-diagram displaying selection of protein measurements used for feature selection in LASSO regression. 34 Somalogic protein measurements displayed a Pearson correlation>0.3 versus the IFNGS and were known to have gene expression inducible by type I IFN through in vitro or in vivo human experiments. B. Average Pearson correlation of type I IFN protein scores predicted through 10 iterations of 5 fold cross validation with the IFNGS varying the number of features input into the LASSO regression model. Optimal value of λ was also chosen through 10 iterations of 5 fold cross validation. Feature selection was restricted to proteins with positive independent associations to the IFNGS. Proteins were scaled to the HD mean and standard deviation prior to fitting the LASSO regression model. C. Regression coefficients from 4 protein signature refit to the IFNα/β 21-gene signature with ordinary least squares regression D. Scatterplot displaying concordance between 4 protein type I IFN signature and the IFNGS. R value calculated using Pearson correlation.

Independent confirmation of IFNPS components by microarray and Rules Based Medicine (RBM). Validation of components of Somalogic measurements of 4 protein type I IFN signature in independent platforms. For each component of the signature, pairwise Spearman correlation between the Somalogic measurements and protein measurements through RBM or paired gene expression measured by microarray are reported. All correlations were significant (p<0.001) except the correlation between Somalogic BLC protein measurements and BLC gene expression measurements reported by the HGU133 Plus 2.0 microarray.

FIG. 3: IFNPS elevated above healthy donors for most IFNGS test-high patients (89%) and also for a subgroup of IFNGS test-low patients (26%). A Density plots displaying prevalence of 21 gene IFNα/β signature in SLE and HD. B. 4 protein type I IFN signature in HD and SLE patients. C. Prevalence of IFNα/β gene signature in HD and SLE patients with low and high prevalence of IFNα/β gene signature. D. Prevalence of type I IFN protein signature in HD and SLE patients with low and high prevalence of IFNα/β gene signature. Statistical comparisons between each group of SLE patients and HD were reported with the Area Under the Curve (AUC) and p-value reported from the Mann-Whitney U Test.

FIG. 6: IFNPS correlates with SLE global disease activity (SLEDAI). Scatterplots displaying correlation between IFNGS and SLEDAI (A) and 4 protein type I IFN signature and SLEDAI in SLE patients. B. AUC of IFNGS and IFNPS in discriminating SLE patients with and without specific SLE symptoms. Threshold for Leukopenia<3000 WBC/μl. P-values reported using Mann-Whitney U test (*** p<0.001, ** p<0.01, * p<0.05, p<0.10).

FIG. 6. cont: IFNPS correlates with SLEDAI in both lymphopenic and non-lymphopenic SLE patients. C. Scatterplot displaying relationship between the IFNGS and the 4 protein type I IFN signature in non-lymphopenic and B. lymphopenic SLE patients. Threshold for Lymphopenia<1000 Lymphocytes/μl. Regression line between both signatures fit using ordinary least squares regression. Correlation coefficient and p-value reported using Spearman's correlation.

1. A method of treating a type I IFN mediated disease in a subject, comprising administering to the subject a therapeutically effective amount of an anti-IFNAR antibody, wherein the patient is identified as having an elevated interferon protein signature (IFNPS) characterised by elevated protein expression of EPHB2, BLC, LAG-3 and IP-10 in the serum compared to a subject not having the type I IFN mediated disease.

2. The method of claim 1, wherein the anti-IFNAR antibody is anifrolumab and a functional derivative thereof.

3. The method of claim 2, comprising administration of 300 mg anifrolumab, optionally wherein the method comprises intravenous administration of 300 mg anifrolumab every 4 weeks.

4. The method of any preceding claim, wherein treatment supresses the IFNPS.

5. The method of any preceding claim, wherein the type I IFN mediated disease is SLE.

6. The method of claim 5, wherein the treatment results in an improvement of the subject's SLE Disease Activity Index (SLEDAI).

7. The method of claim 5 or 6, wherein the treatment results in an improvement of the subject's Cutaneous Lupus Erthematosus Disease Area and Severity Index (CLASI) activity score.

8. The method of any of claims 1 to 4, wherein the IFN mediated disease is myositis.

9. The method of any preceding claim, wherein the subject is identified as not having an elevated IFNGS signature compared to a subject not having the type IFN mediated disease.

10. The method of any preceding claim, wherein treatment decreases the elevated IFNPS signature.

11. A pharmaceutical composition for use in the treatment of a type I interferon-mediated disease in a subject, wherein the pharmaceutical composition comprises a therapeutically effective amount of an anti-IFNAR antibody and wherein the subject is identified as having an elevated IFN protein signature characterised by elevated EPHB2, BLC, LAG-3 and IP-10 protein expression.

12. The pharmaceutical composition for the use of claim 10, wherein the anti-IFNAR antibody is anifrolumab.

13. The pharmaceutical composition of the use of claim 11, wherein the pharmaceutical composition comprises 300 mg anifrolumab.

14. The pharmaceutical composition for the use of claim 13, wherein the use comprises administration of 300 mg of anifrolumab every four weeks.

15. The pharmaceutical composition for the use of claims 10 to 13, wherein treatment supresses the IFNPS.

16. The method of any of claims 10 to 14, wherein the type I IFN mediated disease is SLE.

17. The pharmaceutical composition for the use of claim 15, wherein the treatment results in an improvement of the subject's SLE Disease Activity Index (SLEDAI).

18. The pharmaceutical composition of the use of claims 16 or 17, wherein the treatment results in an improvement of the subject's Cutaneous Lupus Erthematosus Disease Area and Severity Index (CLASI) activity score.

19. The pharmaceutical composition for the use of claim 10, wherein the IFN mediated disease is myositis.

20. The pharmaceutical composition for the use of any of claims 10-19, wherein the subject is identified as not having an elevated IFNGS signature compared to a subject not having the type IFN mediated disease.

21. An in vitro method for detecting elevated IFN activity in a sample isolated from a subject, the in vitro method comprising quantifying EPHB2, BLC, LAG-3 and IP-10 protein expression in the sample and comparing the protein expression in the sample with EPHB2, BLC, LAG-3 and IP-10 protein expression in a sample from a healthy donor.

22. A method of identifying a subject suitable for treatment of a type I interferon-mediated disease or disorder with a therapeutic agent that modulates type I interferon activity, said method comprising detecting an increased level of a first protein in a sample of the subject and an increased level of a second protein in a sample of the subject,
wherein the first protein is EPHB2,
wherein the second protein has a gene expression inducible by type I interferon and displays a Pearson correlation coefficient greater than 0.3 versus a type I interferon gene signature, and
wherein the increased level of the first protein and the increased level of the second protein is relative to:
a) the level of the first protein and the level of the second protein, respectively, in a sample from a tissue or person not having the type I interferon-mediated disease or disorder, or
b) the level of one or more control proteins in a sample of the subject.

23. A method of identifying a subject suitable for treatment of a type I interferon-mediated disease or disorder with a therapeutic agent that modulates type I interferon activity comprising:
i) detecting an increased level of a first protein in a sample of the subject and an increased level of a second protein in a sample of the subject,
wherein the first protein is EPHB2,
wherein the second protein has a gene expression inducible by type I interferon and displays a Pearson correlation coefficient greater than 0.3 versus a type I interferon gene signature, and
wherein the increased level of the first protein and the increased level of the second protein is relative to:
a) the level of the first protein and the level of the second protein, respectively, in a sample from a tissue or person not having the type I interferon-mediated disease or disorder, or
b) the level of one or more control proteins in a sample of the subject; and
ii) administering the therapeutic agent.

24. An anti-type I interferon antibody or an anti-type I interferon receptor antibody that modulates type I interferon activity for use in the treatment of a type I interferon-mediated disease or disorder in a subject, wherein the subject has been identified by detecting an increased level of a first protein in a sample of the subject and an increased level of a second protein in a sample of the subject,
wherein the first protein is EPHB2,
wherein the second protein has a gene expression inducible by type I interferon and displays a Pearson correlation coefficient greater than 0.3 versus a type I interferon gene signature,
and
wherein the increased level of the first protein and the increased level of the second protein is relative to:
a) the level of the first protein and the level of the second protein, respectively, in a sample from a tissue or person not having the type I interferon-mediated disease or disorder,
or
b) the level of one or more control proteins in a sample of the subject.

25. A method of treating a type I interferon-mediated disease or disorder in a subject, the method comprising administering an anti-type I interferon antibody or an anti-type I interferon receptor antibody that modulates type I interferon activity, wherein the subject has been identified by detecting an increased level of a first protein in a sample of the subject and an increased level of a second protein in a sample of the subject,
wherein the first protein is EPHB2,
wherein the second protein has a gene expression inducible by type I interferon and displays a Pearson correlation coefficient greater than 0.3 versus a type I interferon gene signature, and
wherein the increased level of the first protein and the increased level of the second protein is relative to:
a) the level of the first protein and the level of the second protein, respectively, in a sample from a tissue or person not having the type I interferon-mediated disease or disorder,
or
b) the level of one or more control proteins in a sample of the subject.

26. The method of any one of claims 22, 23, or 25, or the antibody for use according to claim 3, further comprising detecting an increased level of at least one other protein in a sample of the subject, wherein the at least one other protein has a gene expression inducible by type I interferon and displays a Pearson correlation coefficient greater than 0.3 versus a type I interferon gene signature; and wherein the increased level of the at least one other protein is relative to:
a) the level of the at least one other protein in a sample from a tissue or person not having the type I interferon-mediated disease or disorder, or
b) the level of one or more control proteins in a sample of the subject.

27. A method of monitoring or prognosing a type I interferon-mediated disease or disorder progression in a subject comprising:
i) identifying a first protein expression level in an initial sample of the subject and at least one other protein expression level in an initial sample of the subject; and
ii) identifying the first protein expression level in a further sample of the subject and the at least one other protein expression level in a further sample of the subject;
wherein an increase in the expression level of the first protein and an increase in the expression level of the at least one other protein in the further sample relative to the initial sample of the subject prognoses disease progression; or
wherein a decrease in the expression level of first protein and a decrease in the expression level of the at least one other protein in the further sample relative to the initial sample of the subject prognoses disease regression;
wherein the first protein is EPHB2; and
wherein the at least one other protein has a gene expression inducible by type I interferon and displays a Pearson correlation coefficient greater than 0.3 versus a type I interferon gene signature.

28. A method of monitoring a type I interferon-mediated disease or disorder progression in a subject receiving treatment with a therapeutic agent that modulates type I interferon activity comprising:
i) identifying a first protein expression level in an initial sample of the subject and at least one other protein expression level in an initial sample of the subject;
ii) identifying the first protein expression level in a further sample of the subject and the at least one other protein expression level in a further sample of the subject;
iii) administering a therapeutic agent that modulates type I interferon activity to the subject, wherein the therapeutic agent is administered prior to step i) or between steps i) and ii); and
iv) comparing the expression levels of the first protein and the at least one other protein in the initial sample of the subject with the expression levels of the first protein and the at least one other protein respectively in the further sample of the subject;
wherein a variance in the expression levels of the first protein and the at least one other protein indicates a level of efficacy of the therapeutic agent that modulates type I interferon activity;
wherein the first protein is EPHB2; and
wherein the at least one other protein has a gene expression inducible by type I interferon and displays a Pearson correlation coefficient greater than 0.3 versus a type I interferon gene signature.

29. A method of identifying a candidate therapeutic agent for treating a type I interferon-mediated disease or disorder comprising:
i) identifying a first protein expression level in an initial sample of the subject and at least one other protein expression level in an initial sample of the subject;
ii) administering the candidate therapeutic agent to the subject;
iii) identifying the first protein expression level in a further sample of the subject and the at least one other protein expression level in a further sample of the subject; and
iv) comparing the expression levels of the first protein and the at least one other protein in the initial sample of the subject with the expression levels of the first protein and the at least one other protein, respectively, in the further sample of the subject;
wherein a variance in the expression levels of the first protein and the at least one other protein comprising a reduction in the up-regulation of the first protein and the at least one other protein expression levels, respectively, indicates that the agent is a candidate therapeutic agent;
wherein the first protein is EPHB2; and
wherein the at least one other protein has a gene expression inducible by type I interferon and displays a Pearson correlation coefficient greater than 0.3 versus a type I interferon gene signature.

30. The method of any one of claims 22, 23, or 25 to 29, or the antibody for use according to claim 3 or claim 5, wherein the second protein and/or the at least one other protein are each independently selected from, ALCAM, Angiopoietin-2, AREG, AXL Receptor Tyrosine Kinase (AXL), b2-Microglobulin, Beta-2-Microglobulin (B2M), C1q, Monocyte Chemotactic Protein 4 (MCP-4), MIP-3b, MCP-1, MCP-3, Monocyte Chemotactic Protein 2 (MCP-2), sCD163, B7-H1, CLM6, CD5L, ST4S6, SCGF-alpha, SCGF-beta, CO8A1, CSF-1, M-CSF R, Cathepsin S, Fractalkine/CX3CL-1, IP-10, I-TAC, BLC, CXCL16, soluble, Monokine Induced by Gamma Interferon (MIG), DLL1, DERM, EMR2, EPHB2, bFGF, VEGF sR3, PHI, TIMD3, Intercellular Adhesion Molecule 1 (ICAM-1), IGFBP-4, IL-13 Ra1, Interleukin-18 (IL-18), IL-18 BPa, Interleukin-1 receptor antagonist (IL-1ra), TCCR, IL-3 Ra, JAG1, KYNU, LAG-3, LDH-H 1, LG3BP, ILT-4, MAPK14, MMP-14, MMP-7, NAGK, Notch-3, Glucocorticoid receptor, PARK7, PD-L2, PDGF-CC, PLPP, NADPH-P450 Oxidoreductase, SAA, a1-Antitrypsin, Sialoadhesin, Siglec-7, SLAF7, Osteopontin, BGH3, TGF-b R III, Tenascin, TNF-a, TNF sR-II, CD30, BAFF, and TS

31. The method of claim 30, or the antibody for use according to claim 30, wherein the level of at least one of (i) the first protein, and (ii) the second protein or the at least one other protein, has an area under the curve (AUC) in SLE v Healthy Donor (HD) of greater 0.5 relative to:
a. the level of the first protein, or the level of the second protein or the at least one other protein, respectively, in a sample from a tissue or person not having the type I interferon-mediated disease or disorder, or
b. the level of one or more control proteins in a sample of the subject.

32. The method of any one of claims 22, 23, or 25 to 31, or the antibody for use according to any one of claims 24, 26, or 30 to 31, wherein the level of at least one of (i) the first protein, and (ii) the second protein or the at least one other protein is which is at least one standard deviation from the Healthy Donor Mean relative to:
a. the level of the first protein, or the level of the second protein or the at least one other protein, respectively, in a sample from a tissue or person not having the type I interferon-mediated disease or disorder, or
b. the level of one or more control proteins in a sample of the subject.

33. The method of any one of claims 30 to 32, or the antibody for use according to any one of claims 30 to 32, wherein the second protein and/or the at least one other protein are each independently selected from BLC, LAG-3 and IP-10.

34. The method of any one of claims 22, 23, or 25 to 33, or the antibody for use according to any one of claims 24, 26, or 30 to 33, wherein the second protein and/or the at least one other protein forms part of a biological pathway independent from the biological pathway of the first protein.

35. The method of any one of claims 22, 23, 25 to 28, or 30 to 34, or the antibody for use according to any one of claims 24, 26, or 30 to 34, wherein the level of at least one of:
the first protein, and
the second protein or the at least one other protein,
is increased by at least 10%.

36. The method of any one of claims 22, 23, 25 to 28, or 30 to 34, or the antibody for use according to any one of claims 24, 26, or 30 to 34, wherein the average of:
the level of the first protein, and
the level of the second protein and/or the level of the at least one other protein,
is increased by at least 10%.

37. The method of any one of claims 22, 23, 25 or 28 to 36, wherein the therapeutic agent is an anti-type I interferon antibody or an anti-type I interferon receptor antibody.

38. The method of claim 37, wherein the anti-type I interferon receptor antibody is anifrolumab.

39. The method of claim 37 or the antibody for use according to any one of claims 24 to 5, or 9 to 15, wherein the anti-type I interferon antibody is sifalumimab.

40. The method of any one of claims 22, 23, or 25 to 39, or the antibody for use according to any one of claims 24, 26, 30 to 37, or 39, wherein the subject is in need of treatment of a type I interferon-mediated disease or disorder selected from systemic lupus erythematosus, discoid lupus, lupus nephritis, dermatomyositis, polymyositis, psoriasis, SSc, vasculitis, sarcoidosis, Sjogren's syndrome, and idiopathic inflammatory myositis.

41. The method of claim 40, or the antibody for use according to claim 40, wherein the subject is in need of treatment of systemic lupus erythematosus.

42. A method of recording the output of the methods of claims 22, 23, or 25 to 41 on a readable medium.

43. A method of treating type I IFN-mediated disease in a patient, comprising selecting the patient, treating the patient
